(19) United States
(12) Patent Application Publication
(10) Pub. No.: US 2024/0366691 A1
(43) Pub. Date: Nov. 7, 2024

(54) COMPOSITIONS AND METHODS FOR TREATING CANCER

(71) Applicant: Vedanta Biosciences, Inc., Cambridge, MA (US)

(72) Inventor: Rajita Menon, Brighton, MA (US)

(73) Assignee: Vedanta Biosciences, Inc., Cambridge, MA (US)

(21) Appl. No.: 18/285,548

(22) PCT Filed: Apr. 5, 2022
(86) PCT No.: PCT/US2022/023427

§ 371 Date: Oct. 4, 2023

Related U.S. Application Data
(60) Provisional application No. 63/170,903, filed on Apr. 5, 2021.

(51) Int. Cl. A61K 35/745; A61K 35/00; A61K 35/741; A61K 39/395; A61P 35/00

(52) U.S. Cl. CPC A61K 35/745; A61K 35/741; A61K 39/3955; A61P 35/00; A61K 2035/115

(57) ABSTRACT

Provided herein are methods and compositions for treating cancer, such as gastric cancer, in a subject involving administering a composition comprising a purified bacterial mixture.

### RELATED APPLICATION

This application claims the benefit under 35 U.S.C. § 119(e) of U.S. provisional application No. 63/170,903, filed Apr. 5, 2021, the entire contents of which are incorporated by reference herein.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Apr. 4, 2022, is named P074570028W000-SEQ-NTJ.txt, and is 34,776 bytes in size.

### BACKGROUND

Cancer is a disease characterized by abnormal cell growth and can be due to any number of factors including diet, consumption of some agents including alcohol and tobacco, infection (e.g., with bacteria such as Helicobacter pylori or viruses such as the Epstein Barr Virus), and environmental factors (e.g., repeated sunburn, exposure to chemicals or radiation). Cancer can be identified by one of the organs, cell types, or areas of the body affected, although the cancer is not limited to the particular area. For example, brain cancer occurs in the brain, as well as other areas, and melanoma occurs in melanocytes in the skin, among other cell types. Colorectal cancer occurs in the colon and in the gastrointestinal tract. Gastric cancer is a cancer of the stomach. Both colorectal and gastric cancers are associated with multiple causes, including environmental risk factors, chronic inflammation, infection with Helicobacter pylori, the presence of gastric polyps, loss of E-cadherin expression, as well as hereditary/genetic factors. In many instances, cancer is asymptomatic until later stages, which may be more difficult to treat with conventional therapies and are associated with poor prognosis.

### SUMMARY

Aspects of the present disclosure provide compositions comprising a purified bacterial mixture comprising one or more bacterial strains selected from the group consisting of Collinsella aerofaciens, Bifidobacterium adolescentis, and Faecalibacterium prausnitzii. In some embodiments, the purified bacterial mixture comprises 2 or 3 of the bacterial strains. In some embodiments, the purified bacterial mixture does not comprise Ruminococcus gnavus, Bacteroides fragilis, and Anaerostipes caccae. In some embodiments, the purified bacterial mixture consists of Collinsella aerofaciens, Bifidobacterium adolescentis, and Faecalibacterium prausnitzii.

In some embodiments, the purified bacterial mixture further comprises one or more bacterial strains selected from the group consisting of Fusobacterium varium, Fusobacterium ulcerans; Fusobacterium sp., Eubacteium limosum, Geminger formicilis, Subdolinogranulum sp.; Ruminococcaceae bacterium; Ruthenibacterium lactatiformans, Phascolarctobacterium faecium, Phascolarctobacterium sp. CAG:207, Parabacteroides johnsonii, Paraprevotella xylaniphila, Parabacteroides distasonis, Alistipes senegalensis, Alistipes timonensis, Bacteroides dorei, Bacteroides fluxus, Phocaeicola dorei, Bacteroides_B dorei, Parabacteroides gordonii, Parabacteroides timonensis, and Bacteroides uniformis.

In some embodiments, the purified bacterial mixture further comprises one or more bacterial strains selected from the group consisting of Bifidobacterium longum, Ruminococcus gnavus, Akkermansia municiniphila, Bacterioides thetaiotaomicron. In some embodiments, the purified bacterial mixture further comprises one or more bacterial strains belonging to a bacterial genus selected from the group consisting of Dorea, Lachnospira, Roseburia, Faecalibacterium, Blautia, Anaerostipes, Butyricicoccus, Gemmiger and Oscillibacter.

In some aspects, the present disclosure provides compositions comprising a purified bacterial mixture comprising one or more bacterial strains selected from the group consisting of Collinsella aerofaciens, Bifidobacterium adolescentis, Faecalibacterium prausnitzii, Bifidobacterium longum, Ruminococcus gnavus, Akkermansia muciniphila, and Bacteroides thetaiotaomicron.

In some aspects, the present disclosure provides compositions comprising a purified bacterial mixture comprising one or more bacterial strains selected from the group consisting of Collinsella aerofaciens, Bifidobacterium adolescentis, Faecalibacteriium prausnitzii and bacterial species belonging to bacterial genera Dorea, Lachnospira, Roseburia, Faecalibacterium, Blautia, Anaerostipes, Butyricicoccus, Gemmiger, and Oscillibacter.

In some aspects, the present disclosure provides compositions comprising a purified bacterial mixture comprising Collinsella aerofaciens, Bifidobacterium adolescentis, Faecalibacteriium prausnitzii and one or more bacterial strains selected from Bifidobacterium longum, Ruminococcus gnavus, Akkermansia muciniphila, and Bacteroides thetaiotaomicron.

In some aspects, the present disclosure provides compositions comprising a purified bacterial mixture comprising Collinsella aerofaciens, Bifidobacterium adolescentis, Faecalibacteriium prausnitzii and one or more bacterial strains belonging to the bacterial genera selected from the group consisting of Dorea, Lachnospira, Roseburia, Faecalibacterium, Blautia, Anaerostipes, Butyricicoccus, Gemmiger, and Oscillibacter.

In some aspects, the present disclosure provides compositions comprising a purified bacterial mixture comprising Collinsella aerofaciens, Bifidobacterium adolescentis, Faecalibacteriium prausnitzii and one or more bacterial strains selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis.

In some aspects, the present disclosure provides compositions comprising a purified bacterial mixture comprising Collinsella aerofaciens, Bifidobacterium adolescentis, Faecalibacteriium prausnitzii; one or more bacterial strains selected from Bifidobacterium longum, Ruminococcus gnavus, Akkermansia muciniphila, and Bacteroides thetaiotaomicron; and one or more bacterial strains selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis.

In some aspects, the present disclosure provides compositions comprising a purified bacterial mixture comprising Collinsella aerofaciens, Bifidobacterium adolescentis, Faecalibacteriium prausnitzii; one or more bacterial strains belonging to the bacterial genera selected from the group consisting of Dorea, Lachnospira, Roseburia, Faecalibacterium, Blautia, Anaerostipes, Butyricicoccus, Gemmiger, and Oscillibacter; and one or more bacterial strains selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis.

In some aspects, the present disclosure provides compositions comprising a purified bacterial mixture comprising Ruminococcus gnavus, Bacteroides fragilis, and Anaerostipes caccae.

In some aspects, the present disclosure provides compositions comprising a purified bacterial mixture comprising Ruminococcus gnavus, Bacteroides fragilis, and Anaerostipes caccae and one or more bacterial strains selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis.

Aspects of the present disclosure provide compositions comprising a purified bacterial mixture comprising one or more bacterial strains comprising 16S rDNA sequences having at least 97% sequence identity to the nucleic acid sequences selected from the group consisting of SEQ ID NOs: 1-3. In some embodiments, the purified bacterial mixture comprises 2 or 3 of the bacterial strains. In some embodiments, the purified bacterial mixture does not comprise a bacterial strain comprising a 16S rDNA sequence having at least 97% sequence identity to the nucleic acid sequence selected from the group consisting of SEQ ID NO: 4-6. In some embodiments, the purified bacterial mixture consists of bacterial strains comprising 16S rDNA sequences having at least 97% sequence identity to the nucleic acid sequences provided by SEQ ID NOs: 1-3.

In some embodiments, the purified bacterial mixture further comprises one or more bacterial strains comprising 16S rDNA sequences having at least 97% sequence identity to the nucleic acid sequences selected from the group consisting of SEQ ID NOs: 10-20.

In some embodiments, the purified bacterial mixture further comprises one or more bacterial strains comprising 16S rDNA sequences having at least 97% sequence identity to the nucleic acid sequences selected from the group consisting of SEQ ID NO: 4, 7, 8, and 9. In some embodiments, the purified bacterial mixture further comprises one or more bacterial strains belonging to a bacterial genera selected from the group consisting of Dorea, Lachnospira, Roseburia, Faecalibacterium, Blautia, Anaerostipes, Butyricicoccus, Gemmiger, and Oscillibacter.

In some aspects, the present disclosure provides compositions comprising a purified bacterial mixture comprising one or more bacterial strains comprising 16S rDNA sequences having at least 97% sequence identity to the nucleic acid sequences selected from the group consisting of SEQ ID NOs: 1-4 and 7-9.

In some aspects, the present disclosure provides compositions comprising a purified bacterial mixture comprising bacterial strains comprising 16S rDNA sequences having at least 97% sequence identity to the nucleic acid sequences of SEQ ID NOs: 1-3, and one or more bacterial strains belonging to a bacterial genera selected from the group consisting of Dorea, Lachnospira, Roseburia, Faecalibacterium, Blautia, Anaerostipes, Butyricicoccus, Gemmiger, and Oscillibacter.

In some aspects, the present disclosure provides compositions comprising a purified bacterial mixture comprising bacterial strains comprising 16S rDNA sequences having at least 97% sequence identity to the nucleic acid sequences of SEQ ID NOs: 1-3; one or more bacterial strains comprising 16S rDNA sequences having at least 97% sequence identity to the nucleic acid sequences of SEQ ID NOs: 4 and 7-9; and one or more bacterial strains comprising 16S rDNA sequences having at least 97% sequence identity to the nucleic acid sequences of SEQ ID NOs: 10-20.

In some aspects, the present disclosure provides compositions comprising a purified bacterial mixture comprising bacterial strains comprising 16S rDNA sequences having at least 97% sequence identity to the nucleic acid sequences of SEQ ID NOs: 1-3; one or more bacterial strains belonging to a bacterial genera selected from the group consisting of Dorea, Lachnospira, Roseburia, Faecalibacterium, Blautia, Anaerostipes, Butyricicoccus, Gemmiger, and Oscillibacter; and one or more bacterial strains comprising 16S rDNA sequences having at least 97% sequence identity to the nucleic acid sequences of SEQ ID NOs: 10-20.

In some aspects, the present disclosure provides compositions comprising a purified bacterial mixture comprising bacterial strains comprising 16S rDNA sequences having at least 97% sequence identity to the nucleic acid sequences of SEQ ID NOs: 4-6.

In some aspects, the present disclosure provides compositions comprising a purified bacterial mixture comprising bacterial strains comprising 16S rDNA sequences having at least 97% sequence identity to the nucleic acid sequences of SEQ ID NOs: 4-6 and one or more bacterial strains comprising 16S rDNA sequences having at least 97% sequence identity to the nucleic acid sequences of SEQ ID NOs: 10-20.

In some aspects, the present disclosure provides compositions comprising a purified bacterial mixture comprising bacterial strains comprising 16S rDNA sequences having at least 97% sequence identity to the nucleic acid sequences of SEQ ID NOs: 1-3, and one or more bacterial strains comprising 16S rDNA sequences having at least 97% sequence identity to the nucleic acid sequences of SEQ ID NOs: 10-20.

In some aspects, the present disclosure provides compositions comprising a purified bacterial mixture comprising bacterial strains comprising 16S rDNA sequences having at least 97% sequence identity to the nucleic acid sequences of SEQ ID NOs: 1-3, and one or more bacterial strains comprising 16S rDNA sequences having at least 97% sequence identity to the nucleic acid sequences selected from the group consisting of SEQ ID NOs: 4 and 7-9.

In some embodiments, one or more of the bacterial strains are not spore-formers. In some embodiments, one or more of the bacterial strains are in spore form. In some embodiments, one or more of the bacterial strains are in vegetative form. In some embodiments, each of the bacterial strains is in vegetative form.

In some embodiments, the bacterial strains originate from one human donor. In some embodiments, the bacterial strains originate from more than one human donor.

In some embodiments, the bacterial strains are lyophilized. In some embodiments, the bacterial strains are spray-dried.

In some embodiments, the pharmaceutical composition comprises between 1×10⁷ and 1×10¹⁰ colony forming units (CFUs) per bacterial strain. In some embodiments, each bacterial strain is present in the composition in the same CFU quantities (e.g., each strain is present at 1×10⁸ CFU).

In some embodiments, the pharmaceutical composition further comprises one or more enteric polymers. In some embodiments, the composition further comprises one or more anticancer agents. In some embodiments, the anticancer agent is a cancer immunotherapy agent. In some embodiments, the cancer immunotherapy agent is an immune checkpoint inhibitor. In some embodiments, the immune checkpoint inhibitor is a PD-1 inhibitor, a PD-L1 inhibitor, or a CTLA-4 inhibitor.

Aspects of the present disclosure provide pharmaceutical compositions comprising any of the compositions described herein and a pharmaceutically acceptable excipient.

In some embodiments, the pharmaceutical composition is in the form of a capsule. In some embodiments, the composition is formulated for oral administration. In some embodiments, the composition is formulated for rectal administration. In some embodiments, the pharmaceutical composition is formulated for delivery to the intestine. In some embodiments, the pharmaceutical composition is formulated for delivery to the colon.

Aspects of the present disclosure provide methods of treating cancer comprising administering to a subject in need thereof any of the compositions described herein in an effective amount. In some embodiments, the cancer is gastric cancer. In some embodiments, the subject is a human subject.

In some embodiments, the subject is administered one or more doses of an antibiotic prior to the composition. In some embodiments, the composition is administered to the subject as one dose. In some embodiments, the composition is administered to the subject more than once. In some embodiments, the composition is administered to the subject as multiple doses.

In some embodiments, the methods further comprise administering to the subject one or more anticancer agent. In some embodiments, the anticancer agent is a chemotherapy agent. In some embodiments, the anticancer agent is a cancer immunotherapy agent. In some embodiments, the cancer immunotherapy agent is an immune checkpoint inhibitor. In some embodiments, the immune checkpoint inhibitor is a PD-1 inhibitor, PD-L1 inhibitor, or CTLA-4 inhibitor.

In some embodiments, the methods further comprise administering to the subject a second composition comprising a purified bacterial mixture comprising one or more bacterial strains. In some embodiments, the second compositions comprises one or more bacterial strains selected from the group consisting of Fusobacterium varium, Fusobacterium ulcerans; Fusobacterium sp., Eubacteium limosum, Geminger formicilis, Subdolinogranulum sp.; Ruminococcaceae bacterium; Ruthenibacterium lactatiformans, Phascolarctobacterium faecium, Phascolarctobacterium sp. CAG:207, Parabacteroides johnsonii, Paraprevotella xylaniphila, Parabacteroides distasonis, Alistipes senegalensis, Alistipes timonensis, Bacteroides dorei, Bacteroides fluxus, Phocaeicola dorei, Bacteroides_B dorei, Parabacteroides gordonii, Parabacteroides timonensis, and Bacteroides uniformis.

These and other aspects of the disclosure, as well as various embodiments thereof, will become more apparent in reference to the drawings and detailed description of the disclosure.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways.

### BRIEF DESCRIPTION OF THE DRAWINGS

The accompanying drawings, which constitute a part of this specification, illustrate several embodiments of the disclosure and together with the description, serve to explain the principles of the disclosure.

FIGS. 1A-1D show data related to the gut microbiome composition and responses of subjects with colorectal cancer, gastric cancer, or melanoma following administration of a purified bacterial composition comprising 11 bacterial strains. FIG. 1A shows the abundance of each of 11 bacterial strains that were administered to Subject A over time, at the indicate time post administration (day 1 (D1), day 3 (D3), day 7 (D7), day 14 (D14), day 21 (D21), day 30 (D30), day 58 (D58), and day 86 (D86). FIG. 1B is a plot of the principal component analysis (PCA) of the association of bacterial genera with the response of a cohort of subjects with gastric cancer. The patients with stable disease (“SD”) after 8 weeks of treatment are indicated in light blue (with asterisks) whereas the patients with progressive disease (“PD”) after 8 weeks of treatment are indicated in pink. As indicated in the circle, Subject A (with stable disease) had a distinct microbiome over time. FIG. 1C shows the response of subjects having colorectal cancer (left panel), gastric cancer (center panel), and melanoma (right panel), at the indicated time post administration of the bacterial compositions (day 1 (D1), day 3 (D3), day 7 (D7), day 14 (D14), day 21 (D21), and day 30 (D30). The patients with SD after 8 weeks of treatment are indicated in light blue (with asterisks) whereas the patients with PD after 8 weeks of treatment are indicated in pink. As indicated in the circle, Subject A, a stable disease patient in the gastric cancer cohort, is indicated in the center panel. FIG. 1D is a bar graph showing the AUC (log(Response Index)) results after 8 weeks of treatment (i.e., 8 weeks of administration of a purified bacterial composition comprising 11 bacterial strains),

FIGS. 2A and 2B show principal component analyses (PCA) correlating the response index/recovery of Subject A over time based on the composition of the gut microbiome. FIG. 2A shows PCA based on the abundance of the response index taxa only. FIG. 2B shows PCA based on all genera present in the gut microbiome. Arrows in FIG. 2B indicate the top 15 genera highly correlated with the principal component.

### DETAILED DESCRIPTION

Cancer is generally characterized by the uncontrolled proliferation of malignant cells. In particular, gastric cancer is characterized by the proliferation of malignant cells and/or formation of tumors in the gastrointestinal tract or gut, which may infiltrate adjacent spaces such as the esophagus or lamina propria. Gastric cancer is broadly classified into two subtypes, intestinal and diffuse, which differ in epidemiology and pathogenesis. Without wishing to be bound by any particular theory, the composition of the gut microbiome is thought to affect the development and progression of gastric cancer. For example, the presence of Helicobacter pylori is associated with the development of the intestinal subtype of gastric cancer. Conversely, the abundance of certain bacterial taxa have been associated with improved outcomes in gastric cancer.

In some embodiments, the compositions described herein contain a purified bacterial mixture comprising bacterial strains that were identified as associated with patient responsiveness to checkpoint inhibitor therapy.

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Provided herein are compositions comprising purified bacterial mixtures comprising one or more bacterial strains selected from Collinsella aerofaciens, Bifidobacterium adolescentis, and Faecalibacterium prausnitzii. In some embodiments, the compositions do not comprise Ruminococcus gnavus, Bacteroides fragilis, and Anaerostipes caccae. Also provided herein are methods for treating cancer (e.g., gastric cancer) in a subject comprising administering compositions comprising purified bacterial mixtures comprising one or more bacterial strains selected from Collinsella aerofaciens, Bifidobacterium adolescentis, and Faecalibacterium prausnitzii.

In some embodiments of the compositions provided herein, the composition includes one or more of the following bacterial strains: Collinsella aerofaciens, Bifidobacterium adolescentis, and Faecalibacterium prausnitzii. In some embodiments, the compositions described herein comprise 2 or 3 of the bacterial strains.

As will be appreciated by one of ordinary skill in the art, a bacterial strain may be closely related to one or more bacterial species. Alternatively or in addition, a bacterial strain may be referred to by one or more bacterial species names, based on changing nomenclature and phylogenetic classification. In some embodiments, the composition comprises a purified bacterial mixture comprising Collinsella aerofaciens. In some embodiments, the composition comprises a purified bacterial mixture comprising Bifidobacterium adolescentis. In some embodiments, the composition comprises a purified bacterial mixture comprising Faecalibacterium prausnitzii. In some embodiments, the composition comprises a purified bacterial mixture comprising Collinsella aerofaciens and Bifidobacterium adolescentis. In some embodiments, the composition comprises a purified bacterial mixture comprising Collinsella aerofaciens and Faecalibacterium prausnitzii. In some embodiments, the composition comprises a purified bacterial mixture comprising Bifidobacterium adolescentis and Faecalibacterium prausnitzii. In some embodiments, the composition comprises a purified bacterial mixture comprising Collinsella aerofaciens Bifidobacterium adolescentis, and Faecalibacterium prausnitzii.

In some embodiments, the composition consists of Collinsella aerofaciens and Bifidobacterium adolescentis. In some embodiments, the composition consists of Collinsella aerofaciens and Faecalibacterium prausnitzii. In some embodiments, the composition consists of Bifidobacterium adolescentis and Faecalibacterium prausnitzii. In some embodiments, the composition consists of Collinsella aerofaciens, Bifidobacterium adolescentis, and Faecalibacterium prausnitzii.

In some embodiments, the composition consists essentially of Collinsella aerofaciens and Bifidobacterium adolescentis. In some embodiments, the composition consists essentially of Collinsella aerofaciens and Faecalibacterium prausnitzii. In some embodiments, the composition consists essentially of Bifidobacterium adolescentis and Faecalibacterium prausnitzii. In some embodiments, the composition consists essentially of Collinsella aerofaciens, Bifidobacterium adolescentis, and Faecalibacterium prausnitzii.

In some embodiments, the composition does not include Ruminococcus gnavus. In some embodiments, the composition does not include Bacteroides fragilis. In some embodiments, the composition does not include Anaerostipes caccae. In some embodiments, the composition does not include Ruminococcus gnavus and Bacteroides fragilis. In some embodiments, the composition does not include Ruminococcus gnavus and Anaerostipes caccae. In some embodiments, the composition does not include Bacteroides fragilis and Anaerostipes caccae. In some embodiments, the composition does not include Ruminococcus gnavus, Bacteroides fragilis, and Anaerostipes caccae.

In some embodiments, the compositions may comprise one or more additional bacterial strains in addition to the bacterial mixture of one or more of Collinsella aerofaciens, Bifidobacterium adolescentis, and Faecalibacterium prausnitzii. Additional bacterial strains that may be included in the compositions described herein can be found, for example, in PCT Publication No. WO 2018/117263, which is incorporated herein by reference in their entirety.

In some embodiments, the compositions further comprise one or more additional bacterial strains selected from Fusobacterium varium, Fusobacterium ulcerans; Fusobacterium sp., Eubacteium limosum, Geminger formicilis, Subdolinogranulum sp.; Ruminococcaceae bacterium; Ruthenibacterium lactatiformans, Phascolarctobacterium faecium, Phascolarctobacterium sp. CAG:207, Parabacteroides johnsonii, Paraprevotella xylaniphila, Parabacteroides distasonis, Alistipes senegalensis, Alistipes timonensis, Bacteroides dorei Bacteroides fluxus, Phocaeicola dorei, Bacteroides_B dorei, Parabacteroides gordonii, Parabacteroides timonensis, and Bacteroides uniformis. In some embodiments, the compositions comprise Collinsella aerofaciens, Bifidobacterium adolescentis, and Faecalibacterium prausnitzii and one or more additional bacterial strains selected from Fusobacterium varium, Fusobacterium ulcerans; Fusobacterium sp., Eubacteium limosum, Geminger formicilis, Subdolinogranulum sp.; Ruminococcaceae bacterium; Ruthenibacterium lactatiformans, Phascolarctobacterium faecium, Phascolarctobacterium sp. CAG:207, Parabacteroides johnsonii, Paraprevotella xylaniphila, Parabacteroides distasonis, Alistipes senegalensis, Alistipes timonensis, Bacteroides dorei Bacteroides fluxus, Phocaeicola dorei, Bacteroides_B dorei, Parabacteroides gordonii, Parabacteroides timonensis, and Bacteroides uniformis.

In some embodiments, the composition further comprises any one or more of the bacterial strains selected from Ruminococcus gnavus, Akkermansia muciniphila, and Bacteroides thetaiotaomicron. In some embodiments, the composition further comprises Ruminococcus gnavus. In some embodiments, the composition further comprises Akkermansia muciniphila. In some embodiments, the composition further comprises Bacteroides thetaiotaomicron. In some embodiments, the composition further comprises Ruminococcus gnavus and Akkermansia muciniphila. In some embodiments, the composition further comprises Akkermansia muciniphila and Bacteroides thetaiotaomicron. In some embodiments, the composition further comprises Ruminococcus gnavus, Akkermansia muciniphila, and Bacteroides thetaiotaomicron.

In some embodiments, the composition further comprises one or more bacterial strains belonging to the bacterial genera selected from the group consisting of Dorea, Lachnospira, Roseburia, Faecalibacterium, Blautia, Anaerostipes, Butyricicoccus, Gemmiger, and Oscillibacter. In some embodiments, the compositions comprise Collinsella aerofaciens, Bifidobacterium adolescentis, and Faecalibacterium prausnitzii and one or more additional bacterial strains selected from the group consisting of Dorea, Lachnospira, Roseburia, Faecalibacterium, Blautia, Anaerostipes, Butyricicoccus, Gemmiger, and Oscillibacter.

An example composition of the present disclosure that may be used, for example, to treat cancer may comprise bacterial strains belonging to species Collinsella aerofaciens, Bifidobacterium adolescentis, Bifidobacterium longum, Faecalibacteriium prausnitzii, Ruminococcus gnavus, Akkermansia muciniphila, and Bacteroides thetaiotaomicron. In some embodiments, an example composition of the present disclosure that may be used, for example, to treat gastric cancer may comprise bacterial strains belonging to species Collinsella aerofaciens, Bifidobacterium adolescentis, and bacterial strains belonging to the bacterial genera Dorea, Lachnospira, Roseburia, Faecalibacterium, Blautia, Anaerostipes, Butyricicoccus, Gemmiger and Oscillibacter.

In some embodiments, the composition comprises a purified bacterial mixture comprising one or more bacterial strains selected from the group consisting of Collinsella aerofaciens, Bifidobacterium adolescentis, Faecalibacterium prausnitzii, Bifidobacterium longum, Ruminococcus gnavus, Akkermansia muciniphila, and Bacteroides thetaiotaomicron.

In some embodiments, the composition comprises a purified bacterial mixture comprising one or more bacterial strains selected from the group consisting of Collinsella aerofaciens, Bifidobacterium adolescentis, Faecalibacteriium prausnitzii and bacterial species belonging to bacterial genera Dorea, Lachnospira, Roseburia, Faecalibacterium, Blautia, Anaerostipes, Butyricicoccus, Gemmiger, and Oscillibacter.

In some embodiments, the composition comprises a purified bacterial mixture comprising Collinsella aerofaciens, Bifidobacterium adolescentis, Faecalibacteriium prausnitzii and one or more bacterial strains selected from Bifidobacterium longum, Ruminococcus gnavus, Akkermansia muciniphila, and Bacteroides thetaiotaomicron.

In some embodiments, the composition comprises a purified bacterial mixture comprising Collinsella aerofaciens, Bifidobacterium adolescentis, Faecalibacteriium prausnitzii and one or more bacterial strains belonging to the bacterial genera selected from the group consisting of Dorea, Lachnospira, Roseburia, Faecalibacterium, Blautia, Anaerostipes, Butyricicoccus, Gemmiger, and Oscillibacter.

In some embodiments, the composition comprises a purified bacterial mixture comprising Collinsella aerofaciens, Bifidobacterium adolescentis, Faecalibacteriium prausnitzii and one or more bacterial strains selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis.

In some embodiments, the composition comprises a purified bacterial mixture comprising Collinsella aerofaciens, Bifidobacterium adolescentis, Faecalibacteriium prausnitzii; one or more (e.g., 1, 2, 3, or 4) bacterial strains selected from Bifidobacterium longum, Ruminococcus gnavus, Akkermansia muciniphila, and Bacteroides thetaiotaomicron; and one or more (e.g., 1, 2, 3, 4, 5, 6, 7, 8, 9, 10, or 11) bacterial strains selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis.

In some embodiments, the composition comprises a purified bacterial mixture comprising Collinsella aerofaciens, Bifidobacterium adolescentis, Faecalibacteriium prausnitzii; one or more (e.g., 1, 2, 3, 4, 5, or more) bacterial strains belonging to the bacterial genera selected from the group consisting of Dorea, Lachnospira, Roseburia, Faecalibacterium, Blautia, Anaerostipes, Butyricicoccus, Gemmiger, and Oscillibacter; and one or more (e.g., 1, 2, 3, 4, 5, 6, 7, 8, 9, 10, or 11) bacterial strains selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis.

In some embodiments, the composition comprises a purified bacterial mixture comprising Ruminococcus gnavus, Bacteroides fragilis, and Anaerostipes caccae.

In some embodiments, the composition comprises a purified bacterial mixture comprising Ruminococcus gnavus, Bacteroides fragilis, and Anaerostipes caccae and one or more (e.g., 1, 2, 3, 4, 5, 6, 7, 8, 9, 10, or 11) bacterial strains selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis.

The exemplary bacterial strains of compositions disclosed herein can also be identified by their 16S rRNA sequences (SEQ ID NOs: 1-3). Identifying bacteria by their sequences furthermore allows for the identification of additional bacterial strains that are identical or highly similar to the exemplified bacteria. For instance, the 16S rRNA sequences of bacterial strains were used to identify the closest relative (based on percent identity) through comparing these sequences with 16S databases. Closely related bacterial relative may also be identified through whole genome sequencing. The bacterial strains having 16S rRNA sequences provided by SEQ ID NOs: 1-3 are most closely related to the following bacterial species: Collinsella aerofaciens, Bifidobacterium adolescentis, and Faecalibacterium prausnitzii. The bacterial strains having 16S rRNA sequences provided by SEQ ID NOs: 4-6 are most closely related to the following bacterial species: Ruminococcus gnavus, Bacteroides fragilis, and Anaerostipes caccae.

Aspects of the disclosure relate to bacterial strains with 16S rDNA sequences that have homology to a nucleic acid sequence of any one of the sequences of the bacterial strains or species described herein. In some embodiments, the bacterial strain has at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8% or 99.9% homology relative to any of the strains or bacterial species described herein over a specified region of nucleic acid or amino acid sequence or over the entire sequence. It would be appreciated by one of skill in the art that the terms “homology” or “percent homology,” may be used interchangeably with “identity” or “percent identity.” In the context of two or more nucleic acid sequences or amino acid sequences, the terms homology or identity refer to a measure of similarity between two or more sequences or portion(s) thereof. The homology may exist over a region of a sequence that is at least about 50 nucleotides in length, or more preferably over a region that is 100 to 500 or 1000 or more nucleotides in length. In some embodiments, the homology exists over the length the 16S rRNA or 16S rDNA sequence, or a portion thereof.

In some embodiments, the compositions comprise a purified bacterial mixture comprising one or more bacterial strains, wherein the one or more bacterial strains comprising 16S rDNA sequences having at least 97% homology with nucleic acid sequences of SEQ ID NO:1, SEQ ID NO:2, or SEQ ID NO:3. In some embodiments, the compositions comprise a purified bacterial mixture comprising one or more bacterial strains, wherein the bacterial strains comprise 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% homology with nucleic acid sequences SEQ ID NO:1, SEQ ID NO:2, or SEQ ID NO:3. In some embodiments, the compositions described herein comprise 2 or 3 of the bacterial strains.

In some embodiments, the compositions comprise a purified bacterial mixture comprising bacterial strains, wherein the bacterial strains comprise 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% homology with nucleic acid sequences SEQ ID NO:1, SEQ ID NO:2, and SEQ ID NO:3.

In some embodiments, the compositions comprise a purified bacterial mixture consisting of bacterial strains comprising 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% homology with nucleic acid sequences SEQ ID NO:1, SEQ ID NO:2, and SEQ ID NO:3.

In some embodiments, the compositions comprise a purified bacterial mixture consisting essentially of bacterial strains comprising 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% homology with nucleic acid sequences SEQ ID NO:1, SEQ ID NO:2, and SEQ ID NO:3.

In some embodiments, the composition does not include a bacterial strain comprising 16S rDNA sequences having at least 95% homology with nucleic acid sequence SEQ ID NO:4. In some embodiments, the composition does not include a bacterial strain comprising 16S rDNA sequences having at least 95% homology with nucleic acid sequence SEQ ID NO:5. In some embodiments, the composition does not include a bacterial strain comprising 16S rDNA sequences having at least 95% homology with nucleic acid sequence SEQ ID NO:6. In some embodiments, the composition does not include bacterial strains comprising 16S rDNA sequences having at least 95% homology with nucleic acid sequences SEQ ID NO:4, SEQ ID NO:5, and SEQ ID NO:5.

Additionally, or alternatively, two or more sequences may be assessed for the identity between the sequences. The terms “identical” or “percent identity” in the context of two or more nucleic acids or amino acid sequences, refer to two or more sequences or subsequences that are the same. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% sequence identity) over a specified region of a nucleic acid or amino acid sequence or over an entire sequence, when compared and aligned for maximum correspondence over a comparison window, or designated region as measured using one of the following sequence comparison algorithms or by manual alignment and visual inspection. Optionally, the identity exists over a region that is at least about 50 nucleotides in length, or more preferably over a region that is 100 to 500 or 1000 or more nucleotides in length. In some embodiments, the identity exists over the length the 16S rRNA or 16S rDNA sequence.

In some embodiments, the compositions comprise a purified bacterial mixture comprising one or more bacterial strains, wherein the one or more bacterial strains comprising 16S rDNA sequences having at least 97% sequence identity with nucleic acid sequences of SEQ ID NO:1, SEQ ID NO:2, or SEQ ID NO:3. In some embodiments, the compositions comprise a purified bacterial mixture comprising one or more bacterial strains, wherein the bacterial strains comprise 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% sequence identity with nucleic acid sequences SEQ ID NO:1, SEQ ID NO:2, or SEQ ID NO:3. In some embodiments, the compositions described herein comprise 2 or 3 of the bacterial strains.

In some embodiments, the compositions comprise a purified bacterial mixture comprising bacterial strains, wherein the bacterial strains comprise 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% sequence identity with nucleic acid sequences SEQ ID NO:1, SEQ ID NO:2, and SEQ ID NO:3.

In some embodiments, the compositions comprise a purified bacterial mixture consisting of bacterial strains comprising 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% sequence identity with nucleic acid sequences SEQ ID NO:1, SEQ ID NO:2, and SEQ ID NO:3.

In some embodiments, the compositions comprise a purified bacterial mixture consisting essentially of bacterial strains comprising 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% sequence identity with nucleic acid sequences SEQ ID NO:1, SEQ ID NO:2, and SEQ ID NO:3.

In some embodiments, the composition does not include a bacterial strain comprising 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% sequence identity with nucleic acid sequence SEQ ID NO:4. In some embodiments, the composition does not include a bacterial strain comprising 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% sequence identity with nucleic acid sequence SEQ ID NO:5. In some embodiments, the composition does not include a bacterial strain comprising 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% sequence identity with nucleic acid sequence SEQ ID NO:6. In some embodiments, the composition does not include bacterial strains comprising 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% sequence identity with nucleic acid sequences SEQ ID NO:4, SEQ ID NO:5, and SEQ ID NO:6.

As described herein, the compositions may comprise one or more additional bacterial strains in addition to the bacterial mixture of one or more bacterial strains comprising 16S rDNA sequences of SEQ ID NOs: 1-3. Additional bacterial strains that may be included in the compositions described herein can be found, for example, in PCT Publication No WO 2018/117263, which is incorporated herein by reference in its entirety.

In some embodiments, the compositions described herein comprise one or more (e.g., 1, 2, 3, 4, 5, 6, 7, 8, 9, 10, 11) bacterial strains comprising 16S rDNA sequences having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9%, or up to 100% sequence identity with nucleic acid sequences SEQ ID NOs: 10-20. In some embodiments, the compositions comprise a purified bacterial mixture comprising bacterial strains comprising 16S rDNA sequences having at least 97% sequence identity with nucleic acid sequences SEQ ID NO:1, SEQ ID NO:2, and SEQ ID NO:3 and one or more bacterial strains comprising 16S rDNA sequences having at least 97% sequence identity with nucleic acid sequences selected from the group consisting of SEQ ID NOs: 10-20.

In some embodiments, the composition further comprises any one or more of the bacterial strains comprising 16S rDNA sequences having at least 97% sequence identity with nucleic acid sequence selected from SEQ ID NOs: 4 and 7-9. In some embodiments, the compositions comprise a purified bacterial mixture comprising bacterial strains comprising 16S rDNA sequences having at least 97% sequence identity with nucleic acid sequences SEQ ID NO:1, SEQ ID NO:2, and SEQ ID NO:3 and one or more bacterial strains comprising 16S rDNA sequences having at least 97% sequence identity with nucleic acid sequences selected from the group consisting of SEQ ID NOs: 4 and 7-9.

In some embodiments, the composition further comprises one or more bacterial strains belonging to a bacterial genera selected from the group consisting of Dorea, Lachnospira, Roseburia, Faecalibacterium, Blautia, Anaerostipes, Butyricicoccus, Gemmiger, and Oscillibacter.

In some embodiments, the composition comprises a purified bacterial mixture comprising one or more bacterial strains comprising 16S rDNA sequences having at least 97% sequence identity to the nucleic acid sequences selected from the group consisting of SEQ ID NOs: 1-4 and 7-9.

In some embodiments, the composition comprises a purified bacterial mixture comprising bacterial strains comprising 16S rDNA sequences having at least 97% sequence identity to the nucleic acid sequences of SEQ ID NOs: 1-3, and one or more bacterial strains belonging to a bacterial genera selected from the group consisting of Dorea, Lachnospira, Roseburia, Faecalibacterium, Blautia, Anaerostipes, Butyricicoccus, Gemmiger, and Oscillibacter.

In some embodiments, the composition comprises a purified bacterial mixture comprising bacterial strains comprising 16S rDNA sequences having at least 97% sequence identity to the nucleic acid sequences of SEQ ID NOs: 1-3; one or more bacterial strains comprising 16S rDNA sequences having at least 97% sequence identity to the nucleic acid sequences of SEQ ID NOs: 4 and 7-9; and one or more bacterial strains comprising 16S rDNA sequences having at least 97% sequence identity to the nucleic acid sequences of SEQ ID NOs: 10-20.

In some embodiments, the composition comprises a a purified bacterial mixture comprising bacterial strains comprising 16S rDNA sequences having at least 97% sequence identity to the nucleic acid sequences of SEQ ID NOs: 1-3; one or more bacterial strains belonging to a bacterial genera selected from the group consisting of Dorea, Lachnospira, Roseburia, Faecalibacterium, Blautia, Anaerostipes, Butyricicoccus, Gemmiger, and Oscillibacter; and one or more bacterial strains comprising 16S rDNA sequences having at least 97% sequence identity to the nucleic acid sequences of SEQ ID NOs: 10-20.

In some embodiments, the composition comprises a a purified bacterial mixture comprising bacterial strains comprising 16S rDNA sequences having at least 97% sequence identity to the nucleic acid sequences of SEQ ID NOs: 4-6.

In some embodiments, the composition comprises a purified bacterial mixture comprising bacterial strains comprising 16S rDNA sequences having at least 97% sequence identity to the nucleic acid sequences of SEQ ID NOs: 4-6 and one or more bacterial strains comprising 16S rDNA sequences having at least 97% sequence identity to the nucleic acid sequences of SEQ ID NOs: 10-20.

In some embodiments, the composition comprises a purified bacterial mixture comprising bacterial strains comprising 16S rDNA sequences having at least 97% sequence identity to the nucleic acid sequences of SEQ ID NOs: 1-3, and one or more bacterial strains comprising 16S rDNA sequences having at least 97% sequence identity to the nucleic acid sequences of SEQ ID NOs: 10-20.

In some embodiments, the composition comprises a purified bacterial mixture comprising bacterial strains comprising 16S rDNA sequences having at least 97% sequence identity to the nucleic acid sequences of SEQ ID NOs: 1-3, and one or more bacterial strains comprising 16S rDNA sequences having at least 97% sequence identity to the nucleic acid sequences selected from the group consisting of SEQ ID NOs: 4 and 7-9.

Additionally, or alternatively, two or more sequences may be assessed for the alignment between the sequences. The terms “alignment” or “percent alignment” in the context of two or more nucleic acids or amino acid sequences, refer to two or more sequences or subsequences that are the same. Two sequences are “substantially aligned” if two sequences have a specified percentage of amino acid residues or nucleotides that are the same (e.g., at least 80%, at least 85%, at least 90%, at least 95%, at least 96%, at least 97%, at least 98%, at least 99%, at least 99.5%, at least 99.6%, at least 99.7%, at least 99.8% or at least 99.9% identical) over a specified region of the nucleic acid or amino acid sequence or over the entire sequence, when compared and aligned for maximum correspondence over a comparison window, or designated region as measured using one of the following sequence comparison algorithms or by manual alignment and visual inspection. Optionally, the alignment exists over a region that is at least about 50 nucleotides in length, or more preferably over a region that is 100 to 500 or 1000 or more nucleotides in length. In some embodiments, the identity exists over the length the 16S rRNA or 16S rDNA sequence.

For sequence comparison, typically one sequence acts as a reference sequence, to which test sequences are compared. Methods of alignment of sequences for comparison are well known in the art. See, e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA (1998) 85:2444, by computerized implementations of these algorithms (GAP, BESTFIT, FASTA, and TFASTA in the Wisconsin Genetics Software Package, Genetics Computer Group. Madison. WI), or by manual alignment and visual inspection (see. e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. (1977) 25:3389-3402, and Altschul et al., J. Mol. Biol. (1990) 215:403-410, respectively.

It should be appreciated that the terms “bacteria” and “bacterial strains” as used herein are interchangeable.

In some embodiments of the compositions provided herein, one or more of the bacterial strains are human-derived bacteria, meaning the one or more bacterial strains were obtained from or identified from a human or a sample therefrom (e.g., a human donor). In some embodiments of the compositions provided herein, all of the bacterial strains are human-derived bacteria. In some embodiments of the compositions provided herein, the bacterial strains are derived from one human donor. In some embodiments of the compositions provided herein, the bacterial strains are derived from more than one human donor.

The bacterial strains used in the compositions provided herein generally are isolated from the microbiome of healthy individuals. In some embodiments, the compositions include strains originating from a single individual. In some embodiments, the compositions include strains originating from multiple individuals. In some embodiments, the bacterial strains are obtained from multiple individuals, isolated and grown up individually. The bacterial compositions that are grown up individually may subsequently be combined to provide the compositions of the disclosure. It should be appreciated that the origin of the bacterial strains of the compositions provided herein is not limited to the human microbiome from a healthy individual. In some embodiments, the bacterial strains originate from a human with a microbiome in dysbiosis. In some embodiments, the bacterial strains used in the compositions provided herein are isolated from the microbiome of a subject who has or is at risk of having cancer. In some embodiments, the bacterial strains used in the compositions provided herein are isolated from the microbiome of a subject who has cancer and is responsive to an anticancer therapy. In some embodiments, the bacterial strains originate from non-human animals or the environment (e.g., soil or surface water). In some embodiments, the combinations of bacterial strains provided herein originate from multiple sources (e.g., human and non-human animals).

In some embodiments of the compositions provided herein, the composition includes one or more anaerobic bacteria. In some embodiments of the compositions provided herein, the composition includes only anaerobic bacteria. In some embodiments of the compositions provided herein, the composition includes one or more facultative anaerobic bacteria. In some embodiments of the compositions provided herein, the composition includes only facultative anaerobic bacteria. In some embodiments of the compositions provided herein, the composition includes one or more obligate anaerobic bacteria. In some embodiments of the compositions provided herein, the composition includes only obligate anaerobic bacteria.

In some embodiments of the compositions provided herein, one or more of the bacterial strains is a spore-former. In some embodiments of the compositions provided herein, one or more of the bacterial strains is in spore form. In some embodiments of the compositions provided herein, one or more of the bacterial strains is a non-spore former.

In some embodiments, the compositions described herein comprise spore forming and non-spore forming bacterial strains. In some embodiments, the compositions described herein comprise spore-forming bacterial strains. In some embodiments, the compositions described herein comprise only non-spore forming bacterial strains. The spore-forming bacteria can be in spore form (i.e., as spores) or in vegetative form (i.e., as vegetative cells). In spore form, bacteria are generally more resistant to environmental conditions, such as heat, acid, radiation, oxygen, chemicals, and antibiotics. In contrast, in the vegetative state or actively growing state, bacteria are more susceptible to such environmental conditions, compared to in the spore form. In general, bacterial spores are able to germinate from the spore form into a vegetative/actively growing state, under appropriate conditions. For instance, bacteria in spore format may germinate when they are introduced in the intestine.

In some embodiments, at least one (e.g., 1, 2, 3, or more) of the bacterial strains in the composition is a non-spore former. In some embodiments, all of the bacterial strains in the composition are non-spore formers. In some embodiments, at least one (e.g., 1, 2, 3, or more) of the bacterial strains in the composition is a spore former. In some embodiments, at least one (e.g., 1, 2, 3, or more) of the bacterial strains in the composition is in spore form. In some embodiments, at least one (e.g., 1, 2, 3, or more) of the bacterial strains in the composition is in vegetative form (as discussed above, spore forming bacteria can also be in vegetative form). In some embodiments, at least one (e.g., 1, 2, 3, or more) of the bacterial strains in the composition is in spore form and at least one (e.g., 1, 2, 3, or more) of the bacterial strains in the composition is in vegetative form. In some embodiments, at least one bacterial strain that is considered able to form spores (i.e., a spore-former) but is present in the composition in vegetative form. In some embodiments, at least one bacterial strain that is considered able to form spores is present in the composition both in spore form and in vegetative form.

It is envisioned that the bacterial strains of the compositions provided herein are alive and will be alive when they reach the target area (e.g., the intestines). Bacterial spores are considered to be alive in this regard. In some embodiments, bacteria that are administered as spores may germinate in the target area (e.g., the intestines). It should further be appreciated that not all of the bacteria are alive and the compositions can include a percentage (e.g., by weight) that is not alive. In addition, in some embodiments, the compositions include bacterial strains that are not alive when administered or at the time when the composition reaches the target area (e.g., the intestines). It is envisioned that non-living bacteria may still be useful by providing some nutrients and metabolites for the other bacterial strains in the composition.

In any of the compositions provided herein, in some embodiments, the bacterial strains are purified. In any of the compositions provided herein, in some embodiments, the bacterial strains are isolated. Any of the bacterial strains described herein may be isolated and/or purified, for example, from a source such as a culture or a microbiota sample (e.g., fecal matter). The bacterial strains used in the compositions provided herein generally are isolated from the microbiome of healthy individuals. However, bacterial strains can also be isolated from individuals that are considered not to be healthy. In some embodiments, the compositions include strains originating from multiple individuals. As used herein, the term “isolated” refers to bacteria that have been separated from one or more undesired component, such as another bacterium or bacterial strain, one or more component of a growth medium, and/or one or more component of a sample, such as a fecal sample. In some embodiments, the bacteria are substantially isolated from a source such that other components of the source are not detected. As also used herein, the term “purified” refers to a bacterial strain or composition comprising such that has been separated from one or more components, such as contaminants. In some embodiments, the bacterial strain is substantially free of contaminants. In some embodiments, one or more bacterial strains of a composition may be independently purified from one or more other bacteria produced and/or present in a culture or a sample containing the bacterial strain. In some embodiments, a bacterial strain is isolated or purified from a sample and then cultured under the appropriate conditions for bacterial replication, e.g., under anaerobic culture conditions. The bacteria that is grown under appropriate conditions for bacterial replication can subsequently be isolated/purified from the culture in which it is grown.

In some embodiments of the compositions provided herein, the composition further comprises one or more anticancer agents. In some embodiments of the compositions provided herein, the anticancer agent is a chemotherapy agent. In some embodiments of the compositions provided herein, the anticancer agent is cancer immunotherapy agent. In some embodiments of the compositions provided herein, the cancer immunotherapy agent is an immune checkpoint inhibitor. In some embodiments, the immune checkpoint inhibitor is a PD-1 inhibitor, PD-L1 inhibitor, or CTLA-4 inhibitor. (See e.g., Vesely M D, Annu Rev Immunol (2011) 29:235-271; Pardoll, Nature Reviews Cancer (2012) 12, 252-264). In some embodiments of the compositions provided herein, the immune checkpoint inhibitor is a PD-1 inhibitor, PD-L1 inhibitor, CTLA-4 inhibitor, IDO1 inhibitor, LAG3 inhibitor, or TIM3 inhibitor.

In some embodiments, the immune checkpoint inhibitor is a PD-1 inhibitor, PD-L1 inhibitor, or CTLA-4 inhibitor. In some embodiments of the compositions provided herein, the immune checkpoint inhibitor is a PD-1 inhibitor. In some embodiments of the compositions provided herein, the immune checkpoint inhibitor is a PD-L1 inhibitor. In some embodiments of the compositions provided herein, the immune checkpoint inhibitor is a CTLA-4 inhibitor.

In some embodiments, the immune checkpoint inhibitor is a PD-1 inhibitor. In some embodiments, the PD-1 inhibitor is nivolumab. In some embodiments, the PD-1 inhibitor is pembrolizumab.

In some embodiments, the immune checkpoint inhibitor is a PD-L1 inhibitor. In some embodiments, the PD-L1 inhibitor is atezolizumab. In some embodiments, the PD-L1 inhibitor is avelumab. In some embodiments, the PD-L1 inhibitor is durvalumab.

In some embodiments, the immune checkpoint inhibitor is a CTLA-4 inhibitor. In some embodiments, the CTLA-4 inhibitor is an anti-CTLA-4 antibody. Examples of anti-CTLA-4 antibodies include, without limitation, ipilimumab, tremelimumab (CP-675,206), 9H10, 4F10, and 9D9. In some embodiments, the CTLA-4 inhibitor is ipilimumab. In some embodiments, the CTLA-4 inhibitor is tremelimumab.

It should further be appreciated that multiple immune checkpoint inhibitors may be used in the compositions and/or methods disclosed herein. For instance, in a non-limiting example, the methods described herein include both a PD-1 inhibitor and a CTLA-4 inhibitor.

The methods described herein involve administering any of the pharmaceutical compositions described herein to a subject in need thereof. As used herein, “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject is a human. In some embodiments, the human subject is a neonatal subject, a pediatric subject, an adolescent subject, an adult subject, or a geriatric subject. In some embodiments, the subject has or is at risk of having gastric cancer.

Any of the compositions described herein may be administered to a subject in a therapeutically effective amount or a dose of a therapeutically effective amount to treat or prevent cancer. In some embodiments, any of the compositions described herein may be administered to a subject in a therapeutically effective amount or a dose of a therapeutically effective amount to treat or prevent gastric cancer. The terms “treat” or “treatment” refer to reducing or alleviating one or more of the symptoms associated with a disease or disorder (e.g., gastric cancer). The terms “prevent” or “prevention” encompass prophylactic administration and may reduce the incidence or likelihood of experiencing a disease or disorder (e.g., cancer, such as gastric cancer).

As used herein, the term “therapeutically effective amount” may be used interchangeably with the term “effective amount.” A therapeutically effective amount or an effective amount of a composition, such as a pharmaceutical composition, as described herein, is any amount that results in a desired response or outcome in a subject, such as those described herein. In some embodiments, the therapeutically effective amount is an amount sufficient to treat cancer, such as gastric cancer.

It should be appreciated that the term “effective amount,” in reference to a composition comprising bacterial strains, may be expressed as the number of bacteria (e.g., bacterial cells) or colony forming units (CFUs) to be administered. It should further be appreciated that the bacteria can multiply once administered. Thus, administration of even a relatively small amount of bacteria may have therapeutic effects.

In some embodiments of the methods provided herein, the subject has cancer. In some embodiments of the methods provided herein, the cancer is gastric cancer, carcinoma, glioma, mesothelioma, melanoma, lymphoma, leukemia, adenocarcinoma, breast cancer, ovarian cancer, cervical cancer, glioblastoma, multiple myeloma, prostate cancer, Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma, Kaposi's sarcoma, multicentric Castleman's disease, AIDS-associated primary effusion lymphoma, neuroectodermal tumors, or rhabdomyosarcoma. In some embodiments of the methods provided herein, the cancer is gastric cancer.

As described herein, gastric cancer is a condition characterized by uncontrolled growth of cells of the gastrointestinal tract, or gut. Gastric cancers are generally classified into two primary subtypes based on histology: intestinal subtype and diffuse subtype (see, e.g., Lauren. Acta Pathol Microbiol Scand. (1965) 64:31-49).

The intestinal subtype of gastric cancer is associated with inflammation that progresses from chronic gastritis. Gastric cancers (gastric adenocarcinomas) of the intestinal subtype occur more frequently in high-risk groups, and are less likely to be inherited. Intestinal subtype gastric cancer is more common in older individuals, and in male than female individuals. The occurrence of the intestinal subtype gastric cancer may be associated with Helicobacter pylori infection. It is thought that inflammation induced by H. pylori causes gastritis and damage to epithelial cells of the gastrointestinal tract, with replacement cells recruited for epithelial repair occasionally dispersing into the lamina propria, becoming a carcinoma. The presence of H. pylori infection may be an indicator that a subject is at risk of developing gastric cancer. See, e.g., Carcas. J Carcinog. (2014) 13:14.

The diffuse subtype of gastric cancer is may be considered more aggressive than those of the intestinal subtype with poor overall prognosis compared to the intestinal subtype. Diffuse subtype of gastric cancer may be associated with loss of E-cadherin expression in cells rather than H. pylori infection. E-cadherin, encoded by the CDH1 gene, is a transmembrane protein that is required for cell-cell adhesion complex formation. Loss of E-cadherin expression interferes with cell-cell adhesion, allowing cancerous cells to invade the gastric wall and adjacent spaces, such as the duodenum and esophagus. E-cadherin deficiency may be spontaneous, if cells develop or inherit one or more mutations that interfere with E-cadherin function or expression. CDH1 germline mutations, as well as a family history of gastric cancer, may be indicative of risk of developing gastric cancer. See, e.g., Carcas. J Carcinog (2014) 13:14.

Gastric cancers may also be classified based on the stage of cancer severity, for example using the TNM staging system associated with prognosis from the International Union Against Cancer (UICC). The TNM staging system includes factors such as the size of a tumor, spread to nearby lymph nodes, and metastasis. The gastric cancer may be of stage 0, 1A, 1B, II, IIIA, IIIB, or IV.

In many cases, gastric cancer is asymptomatic in early stages and thus may not be diagnosed until advanced stages, when treatment may be difficult and prognosis poor. Common methods of detecting and diagnosing gastric cancer include esophagogastroduodenoscopy and biopsy of the gut of subjects in which gastric cancer is suspected, with histology contributing to determination of the subtype of the cancer. Current methods of treating gastric cancer include, for example, surgical removal of tumors and/or resection of the gastrointestinal tract, tumor ablation (endoscopic palliation), radiation, and chemotherapy. Monoclonal antibodies targeting HER2 or VEGFR2 are currently approved for the treatment of some gastric cancers and have shown some benefit to survival in patients with cancers expressing HER2 or VEGFR2, respectively.

In some embodiments, the subject has received one or more anticancer therapies prior to or concurrently with administration of any one the compositions described herein.

Any of the methods described herein may be for the treatment of cancer in a subject (e.g., gastric cancer). As used herein, methods of treating gastric cancer involve relieving and/or alleviating at least one symptom associated with gastric cancer, slowing or reversing the gastric cancer progression, or improving the prognosis of gastric cancer.

In some embodiments, the subject has or is at risk of having or developing cancer, such as gastric cancer. In some embodiments, the subject has or is at risk of having an H. pylori infection. H. pylori infection is characterized by colonization of the gut by Helicobacter pylori. Methods of diagnosing H. pylori infection are well known in the art and include culture-based methods and detection of a nucleic acid with at least 97% sequence identity to the 16S rDNA sequence of H. pylori in a sample obtained from the stomach of a subject. In some embodiments, the subject has or is at risk of having a mutation in a CDH1 gene.

In some embodiments, the bacterial strains of the compositions provided herein can treat and/or prevent cancer, e.g., gastric cancer, due to the synergy between the bacterial strains of the composition.

In some embodiments of the compositions provided herein, the composition induces proliferation and/or accumulation of CD8+ T-cells. In some embodiments, the bacterial strains of the compositions provided herein can induce proliferation and/or accumulation of CD8+ T-cells, because of synergy between the bacterial strains. Thus, without being limiting to a specific mechanism, in some embodiments, the combination of the bacterial strains of the compositions provided herein act synergistically in the induction of proliferation and/or accumulation of CD8+ T-cells because the combination of the strains is particularly well-suited to generate metabolites and/or cellular signals that stimulate the induction of proliferation and/or accumulation of CD8+ T-cells. The bacterial compositions may do so, for instance through the use of nutrients in the intestinal tract (e.g., the colon or the cecum), and/or metabolic interactions that result in metabolites and/or cellular signals that stimulate the induction of proliferation and/or accumulation of CD8+ T-cells. In addition, without being limiting to a specific mechanism, in some embodiments, the combination of the bacterial strains of the compositions provided herein act synergistically in the induction of proliferation and/or accumulation of CD8+ T-cells because the combination of the strains is superior in engrafting specific niches in the intestinal tract (e.g., the colon or the cecum) that will result in the induction of proliferation and/or accumulation of CD8+ T-cells (e.g., by providing a favorable microenvironment). In some embodiments, the combination of the bacterial strains of the compositions provided herein act synergistically in the induction of proliferation and/or accumulation of CD8+ T-cells because the combination of the strains is particularly well-suited to generate metabolites and/or cellular signals that stimulate the induction of proliferation and/or accumulation of CD8+ T-cells, and the combination is well suited to engraft in specific niches, that result in localization of the metabolites and/or cellular signals to a target for the induction of proliferation and/or accumulation of CD8+ T-cells.

As described herein, any of the pharmaceutical compositions described herein may be administered to a subject in one dose or in multiple doses (e.g., initial administration), which may be followed by one or more additional doses of any of the pharmaceutical compositions described herein. In some embodiments, any of pharmaceutical composition described herein may be administered to a subject in one dose or in multiple doses in an initial administration, followed by one or more additional doses of a pharmaceutical composition comprising the same one or more bacterial strains as the pharmaceutical composition of the initial administration. In some embodiments, any of pharmaceutical composition described herein may be administered to a subject in one dose or in multiple doses in an initial administration, followed by one or more additional doses of a pharmaceutical composition comprising more total bacteria (colony-forming units) relative to the initial administration of the pharmaceutical composition. In some embodiments, any of pharmaceutical composition described herein may be administered to a subject in one dose or in multiple doses in an initial administration, followed by one or more additional doses of a pharmaceutical composition comprising fewer total bacteria (colony-forming units) relative to the initial administration of the pharmaceutical composition. In some embodiments, the initial administration includes at least 1, 2, 3, 4, 5, 6, 7, 8, 9, 10, 11, 12, 13, 14, 15 or more doses of any of the pharmaceutical compositions described herein. In some embodiments, the additional administration includes at least 1, 2, 3, 4, 5, 6, 7, 8, 9, 10, 11, 12, 13, 14, 15 or more doses of any of the pharmaceutical compositions described herein. In some embodiments, the initial administration comprises two doses of any of the pharmaceutical composition and the additional administration comprises three doses of any of the pharmaceutical compositions described herein.

In some embodiments, the subject has not received a dose of an antibiotic prior to administration of the bacterial composition. In some embodiments, the subject has not been administered an antibiotic at least 1, at least 2, at least 3, at least 5, at least 10, at least 15, at least 20, at least 25, at least 30, at least 60, at least 90, at least 120, at least 180 or at least 360 days prior to administration of the compositions provided herein. In some embodiments, the subject is treated with an amount of antibiotics sufficient to allow for the grafting of the one or more strains of the bacterial compositions provided herein.

In some embodiments, the dosing regimen entails administration of multiple doses of any of the compositions described herein. In some embodiments, the composition is administered orally to the subject once, twice, 3 times, 4 times, 5 times, 6 times, 7 times, 8 times, 9 times, at least 10 times, at least 11 times, at least 12 times, at least 13 times, at least 14 times, or more. In some embodiments, any of the compositions described herein are administered to the subject in multiple doses at a regular interval, such as every day, every 2 days, every 3 days, every 4 days, every 5 days, every 6 days, every week, every 2 weeks, every 4 weeks, every month, every 2 months, every 3 months, every 4 months, every 5 months, every 6 months, or more. In some embodiments, one dose of any of the compositions described herein is administered and a second dose of the composition is administered the following day (e.g., consecutive day). In some embodiments, one dose of any of the compositions described herein is administered and each of the additional doses of the composition are administered on consecutive days (e.g., first dose on day 1, second dose of day 2, third dose on day 3, etc.).

In some embodiments, the subject is administered a single dose of an antibiotic prior to the administration of any of the bacterial compositions described herein. In some embodiments, the subject is administered multiple doses of an antibiotic prior to the administration of any of the bacterial compositions described herein. In some embodiments, the subject is administered at least 2, 3, 4, 5 or more doses of an antibiotic prior to the administration of any of the bacterial compositions described herein. In some embodiments, the subject is administered a dose of an antibiotic at substantially the same time as the administration of any of the bacterial compositions described herein. Examples of antibiotics that can be administered include, without limitation, kanamycin, gentamicin, colistin, metronidazole, vancomycin, clindamycin, fidaxomicin, penicillin, streptomycin, and cefoperazone.

In some embodiments in any one the methods provided herein, a subject is evaluated for the presence of one or more of the bacterial strains of the compositions described herein in the microbiome. In some embodiments, if the subject does not have, or only has a low level of one or more of the bacterial strains of the bacterial compositions described herein in the microbiome, any one of the compositions provided herein, or one or more further doses of any one of the compositions provided herein, may be administered.

In some embodiments, the subject is evaluated for the presence of and/or abundance of one or more bacterial strains of the bacterial compositions described herein in the microbiome. In some embodiments, if one or more bacterial strains of the compositions are detected at a level above a threshold level, no further compositions are administered to the subject. In some embodiments, if one or more bacterial strains of the compositions colonize the subject to a level above a threshold level, no further compositions are administered to the subject.

In some embodiments, any of the methods described herein may further comprise administering vancomycin to the subject prior to administration of the pharmaceutical compositions described herein. In some embodiments, the method does not comprise administering an antibiotic to the subject prior to administration of the pharmaceutical compositions described herein. In some embodiments, the method does not comprise administering vancomycin to the subject prior to administration of the pharmaceutical compositions described herein. Vancomycin administration has been found to alter the composition of human gut microbiota. See, e.g., Reijnders et al. Cell Metabolism (2016) 24(1): 63-72. Without wishing to be bound by any particular theory, it is thought that administration of vancomycin may aid engraftment of the bacterial strain(s) of the pharmaceutical compositions described herein, for example by removing other microbes present in the gastrointestinal tract.

In some embodiments, the antibiotic (e.g., vancomycin) is administered to the subject once, as a single dose. In some embodiments, the antibiotic (e.g., vancomycin) is administered to the subject in multiple doses. In some embodiments, the antibiotic (e.g., vancomycin) is administered to the subject in at least 2, 3, 4, 5, 6, 7, 8, 9, 10, 11, 12, 13, 14, 15 or more doses. The multiple doses of the antibiotic (e.g., vancomycin) may be administered to the subject at regular intervals prior to administering any of the pharmaceutical compositions described herein. In some embodiments, each of the multiple doses of the antibiotic (e.g., vancomycin) are administered on consecutive days (e.g., first dose on day 1, second dose of day 2, third dose on day 3, etc.). In some embodiments, the antibiotic (e.g., vancomycin) is administered to the subject for 2, 3, 4, 5, 6, 7, 8, 9, 10, 11, 12, 13, 14, 15 or more consecutive days. In some embodiments, the antibiotic (e.g., vancomycin) is administered to the subject each day for three consecutive days. In some embodiments, the antibiotic (e.g., vancomycin) administered to the subject each day for five consecutive days. In some embodiments, the antibiotic (e.g., vancomycin) administered to the subject each day for seven consecutive days. In some embodiments, the antibiotic (e.g., vancomycin) administered to the subject for one day. In any of the embodiments described herein, a subject may be administered one or more doses of a first antibiotic followed by one or more doses of a second antibiotic.

In some embodiments, the disclosure provides methods comprising administering one or more antibiotics to the subject and subsequently administering any of the bacterial compositions to the subject once, twice, 3 times, 4 times, 5 times, 6 times, 7 times, 8 times, 9 times, or at least 10 times, or more.

In some embodiments, the antibiotic (e.g., vancomycin) is administered according to a pulse tapered regime. See e.g., Sirbu et al., Clinical Infectious Diseases (2017) 65: 1396-1399.

In some embodiments, the antibiotic (e.g., vancomycin) is administered to the subject at least 1, 2, 3, 4, 5, 6, 7 days or more prior to administration of the pharmaceutical compositions described herein. In some embodiments, administration of antibiotic (e.g., vancomycin) is terminated at least one day (e.g., 1, 2, 3, 4, 5, or more) prior to administration of any of the pharmaceutical compositions described herein.

It should be appreciated, in some embodiments, that any of the vancomycin doses or administration regimens may be combined with any of the pharmaceutical composition doses or administration regimens provided herein.

In some embodiments, administration of an antibiotic (e.g., vancomycin) followed by the administration of a single dose or multiple doses of the pharmaceutical composition results in an increase in the rate of engraftment of the initial amount of the bacterial strains of the pharmaceutical composition in the microbiome of the subject compared to the administration of a pharmaceutical composition without the administration of the antibiotic.

In some embodiments, administration of an antibiotic (e.g., vancomycin) followed by the administration of multiple doses of the pharmaceutical composition results in an increase in the abundance of bacterial strains of the pharmaceutical composition in the microbiome of the subject (engraftment) compared to the administration of a single dose of the pharmaceutical composition. In some embodiments, the disclosure provides methods comprising the administration of a pharmaceutical composition provided herein, wherein the administration of multiple doses of the pharmaceutical composition increases the abundance of bacterial strains in the microbiota of the subject (engraftment) of the pharmaceutical composition in the microbiome of the subject compared to the administration of a single dose of the pharmaceutical composition.

In some embodiments of the methods provided herein, the method further includes administering one or more anticancer agents. In some embodiments of the methods provided herein, the anticancer agent is a chemotherapy agent. In some embodiments of the methods provided herein, the anticancer agent is a cancer immunotherapy agent.

In some embodiments of the methods provided herein, the cancer immunotherapy agent is an immune checkpoint inhibitor, such as any of the immune checkpoint inhibitors described herein. In some embodiments of the methods provided herein, the immune checkpoint inhibitor is a PD-1 inhibitor, PD-L1 inhibitor, or CTLA-4 inhibitor. In some embodiments of the methods provided herein, the immune checkpoint inhibitor is a PD-1 inhibitor. In some embodiments of the methods provided herein, the immune checkpoint inhibitor is a CTLA-4 inhibitor.

In some embodiments of the methods provided herein, the cancer immunotherapy agent is a cancer vaccine that acts to increase the response of a subject's immune system to cancer cells. For example, cancer vaccines include cancer antigen(s) that act to induce or stimulate an immune response against cells bearing the cancer antigen(s). The immune response induced or stimulated can include an antibody (humoral) immune response and/or a T-cell (cell-mediated) immune response. CD8+ T-cells can differentiate into cytotoxic T-cells that kill target cells bearing the antigen recognized by CD8+ T-cells. Induction of CD8+ T-cells can, therefore, enhance the immune response to cancer antigens provided in a cancer vaccine.

In some embodiments of the methods provided herein, the method further comprises administering a second composition comprising a purified bacterial mixture comprising bacterial strains to the subject. In some embodiments, the method further involves administering a second compositions comprising bacterial strains selected from Fusobacterium varium, Fusobacterium ulcerans; Fusobacterium sp., Eubacteium limosum, Geminger formicilis, Subdolinogranulum sp.; Ruminococcaceae bacterium; Ruthenibacterium lactatiformans, Phascolarctobacterium faecium, Phascolarctobacterium sp. CAG:207, Parabacteroides johnsonii, Paraprevotella xylaniphila, Parabacteroides distasonis, Alistipes senegalensis, Alistipes timonensis, Bacteroides dorei Bacteroides fluxus, Phocaeicola dorei, Bacteroides_B dorei, Parabacteroides gordonii, Parabacteroides timonensis, and Bacteroides uniformis.

In some embodiments, the methods described herein may involve subjecting the subject to a bowel lavage (bowel irrigation, whole bowel irrigation, gastrointestinal lavage, gastric lavage) prior to administration of the compositions described herein. In some embodiments, a bowel lavage may remove or aid in removing microbiota from the gastrointestinal tract of the subject, creating a niche for the bacterial strains of the compositions described herein. In some embodiments, the bowel lavage may be an oral bowel lavage or a rectal bowel lavage.

Methods of performing a bowel lavage are known in the art, and generally involve the rapid administration of large volumes of a solution, such as polyethylene glycol or a balanced electrolyte solution. A rectal bowel lavage can involve the administration of a solution or a suppository containing the pharmaceutical composition. A bowel lavage may be performed under doctor supervision, hospitalization, or at home.

Any of the compositions described herein, including the pharmaceutical compositions and food products comprising the compositions, may contain bacterial strains in any form, for example in an aqueous form, such as a solution or a suspension, embedded in a semi-solid form, in a powdered form or freeze dried form. In some embodiments, the composition or the bacterial strains of the composition are lyophilized. In some embodiments, a subset of the bacterial strains in a composition is lyophilized. Methods of lyophilizing compositions, specifically compositions comprising bacteria, are well known in the art. See, e.g., U.S. Pat. Nos. 3,261,761; 4,205,132; PCT Publications WO 2014/029578 and WO 2012/098358, herein incorporated by reference in their entirety. The bacteria may be lyophilized as a combination and/or the bacteria may be lyophilized separately and combined prior to administration. A bacterial strain may be combined with a pharmaceutical excipient prior to combining it with the other bacterial strain or multiple lyophilized bacteria may be combined while in lyophilized form and the mixture of bacteria, once combined may be subsequently be combined with a pharmaceutical excipient. In some embodiments, the bacterial strain is a lyophilized cake. In some embodiments, the compositions comprising the one or more bacterial strains are a lyophilized cake.

In some embodiments, one or more of the bacterial strains of the compositions, including pharmaceutical compositions and food products, has been spray-dried. In some embodiments, a subset of the bacterial strains is spray-dried. The process of spray-drying refers to production of dry powder from a liquid comprising bacterial compositions (See, e.g., Ledet, et al., Spray Draying of Pharmaceuticals in “Lyophilized Biologics and Vaccines” pages 273-294, Springer). In general, the process involves rapidly drying the bacterial compositions with a hot gas. A bacterial strain may be combined with a pharmaceutical excipient prior to combining it with the other bacterial strains or multiple spray-dried bacterial strains may be combined while in spray-dried form and the mixture of bacterial strains, once combined, may be subsequently combined with a pharmaceutical excipient.

The bacterial strains of the composition can be manufactured using fermentation techniques well known in the art. In some embodiments, the active ingredients are manufactured using anaerobic fermenters, which can support the rapid growth of anaerobic bacterial strains. The anaerobic fermenters may be, for example, stirred tank reactors or disposable wave bioreactors. Culture media such as BL media and EG media, or similar versions of these media devoid of animal components, can be used to support the growth of the bacterial species. The bacterial product can be purified and concentrated from the fermentation broth by traditional techniques, such as centrifugation and filtration and can optionally be dried and lyophilized by techniques well known in the art.

In some embodiments, the live bacterial product may be formulated for administration as a pharmaceutical composition. The term “pharmaceutical composition” as used herein means a product that results from the mixing or combining of at least one active ingredient, such as any of the bacterial strains described herein, and one or more inactive ingredients, which may include one or more pharmaceutically acceptable excipient.

An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions can be prepared in accordance with methods well known and routinely practiced in the art (see e.g., Remington: The Science and Practice of Pharmacy, Mack Publishing Co. 20th ed. 2000). The pharmaceutical compositions described herein may further comprise any carriers or stabilizers in the form of a lyophilized formulation or an aqueous solution. Acceptable excipients, carriers, or stabilizers may include, for example, buffers, antioxidants, preservatives, polymers, chelating reagents, and/or surfactants. Pharmaceutical compositions are preferably manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like. In some embodiments, the pharmaceutical compositions can be used by injection, such as by intravenous, intramuscular, subcutaneous, or intradermal administration.

In some embodiments, the compositions comprising bacterial strains are formulated for oral delivery. In some embodiments, the bacteria are formulated for delivery to the intestines (e.g., the small intestine and/or the colon). In some embodiments, the bacteria are formulated with an enteric coating that increases the survival of the bacteria through the harsh environment in the stomach. The enteric coating is one which resists the action of gastric juices in the stomach so that the bacteria which are incorporated therein will pass through the stomach and into the intestines. The enteric coating may readily dissolve when in contact with intestinal fluids, so that the bacteria enclosed in the coating will be released in the intestinal tract. Enteric coatings may consist of polymer and copolymers well known in the art, such as commercially available EUDRAGIT (Evonik Industries). See e.g., Zhang, AAPS PharmSciTech (2016) 17 (1), 56-67.

The compositions comprising bacteria may also be formulated for rectal delivery to the intestine (e.g., the colon). Thus, in some embodiments, the bacterial compositions may be formulated for delivery by suppository, colonoscopy, endoscopy, sigmoidoscopy or enema. A pharmaceutical preparation or formulation and particularly a pharmaceutical preparation for oral administration, may include an additional component that enables efficient delivery of the compositions of the disclosure to the intestine (e.g., the colon). A variety of pharmaceutical preparations that allow for the delivery of the compositions to the intestine (e.g., the colon) can be used. Examples thereof include pH-sensitive compositions, more specifically, buffered sachet formulations or enteric polymers that release their contents when the pH becomes alkaline after the enteric polymers pass through the stomach. When a pH-sensitive composition is used for formulating the pharmaceutical preparation, the pH-sensitive composition is preferably a polymer whose pH threshold of the decomposition of the composition is between about 6.8 and about 7.5. Such a numeric value range is a range in which the pH shifts toward the alkaline side at a distal portion of the stomach, and hence is a suitable range for use in the delivery to the colon. It should further be appreciated that each part of the intestine (e.g., the duodenum, jejunum, ileum, cecum, colon and rectum), has different biochemical and chemical environment. For instance, parts of the intestines have different pHs, allowing for targeted delivery by compositions that have a specific pH sensitivity. Thus, the compositions provided herein may be formulated for delivery to the intestine or specific parts of the intestine (e.g., the duodenum, jejunum, ileum, cecum, colon and rectum) by providing formulations with the appropriate pH sensitivity. See e.g., Villena et al., Int J Pharm (2015) 487 (1-2): 314-9.

Also within the scope of the present disclosure are pharmaceutical compositions for administration by additional or alternative routes. In some embodiments, the pharmaceutical compositions are formulated for sublingual administration. In some embodiments, the pharmaceutical compositions are formulated for administration by injection.

In some embodiments, a pharmaceutical composition may include an additional component that enables efficient delivery of the compositions of the disclosure to a desired site, such as the gastrointestinal tract (e.g., the colon).

Another embodiment of a pharmaceutical preparation useful for delivery of the compositions to the intestine (e.g., the colon) is one that ensures the delivery to the colon by delaying the release of the contents (e.g., the bacterial strains) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Additional examples of pharmaceutical compositions that allow for the delivery to the intestine (e.g., the colon) include bioadhesive compositions which specifically adhere to the colonic mucosal membrane (for example, a polymer described in the specification of U.S. Pat. No. 6,368,586) and compositions into which a protease inhibitor is incorporated for protecting particularly a biopharmaceutical preparation in the gastrointestinal tracts from decomposition due to an activity of a protease.

Another example of a system enabling the delivery to the intestine (e.g., the colon) is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

A further example of a system enabling the delivery of a composition to the intestine (e.g., the colon), is a composition that includes a coating that can be removed by an enzyme present in the gut (e.g., the colon), such as, for example, a carbohydrate hydrolase or a carbohydrate reductase. Such a system is not particularly limited, and more specific examples thereof include systems which use food components such as non-starch polysaccharides, amylose, xanthan gum, and azopolymers.

The compositions provided herein can also be delivered to specific target areas, such as the intestine, by delivery through an orifice (e.g., a nasal tube) or through surgery. In addition, the compositions provided herein that are formulated for delivery to a specific area (e.g., the cecum or the colon), may be administered by a tube (e.g., directly into the small intestine). Combining mechanical delivery methods such as tubes with chemical delivery methods such as pH specific coatings, allow for the delivery of the compositions provided herein to a desired target area (e.g., the cecum or the colon).

The compositions comprising bacterial strains are formulated into pharmaceutically acceptable dosage forms by conventional methods known to those of skill in the art. Dosage regimens are adjusted to provide the optimum desired response (e.g., the prophylactic or therapeutic effect). In some embodiments, the dosage form of the composition is a tablet, pill, capsule, powder, granules, solution, or suppository. In some embodiments, the pharmaceutical composition is formulated for oral administration. In some embodiments, the pharmaceutical composition is formulated such that the bacteria of the composition, or a portion thereof, remain viable after passage through the stomach of the subject. In some embodiments, the pharmaceutical composition is formulated for rectal administration, e.g., as a suppository. In some embodiments, the pharmaceutical composition is formulated for delivery to the intestine or a specific area of the intestine (e.g., the colon) by providing an appropriate coating (e.g., a pH specific coating, a coating that can be degraded by target area specific enzymes, or a coating that can bind to receptors that are present in a target area).

Dosages of the active ingredients in the pharmaceutical compositions disclosed herein can be varied so as to obtain an amount of the active ingredient which is effective to achieve the desired pharmaceutical response for a particular subject, composition, and mode of administration, without being toxic or having an adverse effect on the subject. The selected dosage level depends upon a variety of factors including the activity of the particular compositions employed, the route of administration, the time of administration, the duration of the treatment, other drugs, compounds and/or materials used in combination with the particular compositions employed, the age, sex, weight, condition, general health and prior medical history of the subject being treated, and like factors.

A physician, veterinarian or other trained practitioner, can start doses of the pharmaceutical composition at levels lower than that required to achieve the desired therapeutic effect and gradually increase the dosage until the desired effect is achieved. In general, effective doses of the compositions, for the prophylactic or therapeutic treatment of groups of people as described herein vary depending upon many different factors, including routes of administration, physiological state of the subject, whether the subject is human or an animal, other medications administered, and the therapeutic effect desired. Dosages need to be titrated to optimize safety and efficacy. In some embodiments, the dosing regimen entails oral administration of a dose of any of the compositions described herein. In some embodiments, the dosing regimen entails oral administration of multiple doses of any of the compositions described herein. In some embodiments, the composition is administered orally the subject once, twice, 3 times, 4 times, 5 times, 6 times, 7 times, 8 times, 9 times, or at least 10 times. In some embodiments, any of the compositions described herein are administered the subject in multiple doses at a regular interval, such as every 2 weeks, every month, every 2 months, every 3 months, every 4 months, every 5 months, every 6 months, or more.

The compositions, including the pharmaceutical compositions disclosed herein, include compositions that contain selected bacterial strains. The amount of bacteria, including the amount of bacteria cells of each of the bacterial strains, in the compositions, including pharmaceutical compositions, may be expressed in weight, number of bacteria and/or CFUs (colony forming units). In some embodiments, the compositions, including pharmaceutical compositions, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more of each of the bacterial strains per dosage amount. In some embodiments, the compositions, including pharmaceutical compositions, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more total bacterial cells per dosage amount. It should further be appreciated that bacteria of each of the bacterial strains may be present in different amounts. Thus, for instance, as a non-limiting example, composition may include 10³ of bacteria A, 10⁴ of bacteria B, and 10⁶ of bacteria C. In some embodiments, compositions, including pharmaceutical composition, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more bacterial cells or CFUs of each of the bacterial strains per dosage amount. In some embodiments, compositions, including pharmaceutical compositions, comprise about 10¹, about 10², about 10³, about 10⁴, about 10⁵, about 10⁶, about 10⁷, about 10⁸, about 10⁹, about 10¹⁰, about 10¹¹, about 10¹², about 10¹³ or more bacterial cells or CFUs in total for all of the bacterial strains combined per dosage amount. As discussed above, bacteria of each of the bacterial strains may be present in different amounts. In some embodiments, the compositions, including pharmaceutical compositions, contain about 10⁻⁷, about 10⁻⁶, about 10⁻⁵, about 10⁻⁴, about 10⁻³, about 10⁻², about 10⁻¹ or more grams of bacteria (bacterial cells or CFU) of each of the bacterial strains in the composition per dosage amount. In some embodiments, the compositions, including pharmaceutical compositions, contain about 10⁻⁷, about 10⁻⁶, about 10⁻⁵, about 10⁻⁴, about 10-3, about 10⁻², about 10⁻¹ or more grams of bacteria (bacterial cells or CFU) in total for all of the bacterial strains combined per dosage amount.

In some embodiments, the dosage amount is one administration device (e.g., one table, pill or capsule). In some embodiments, the dosage amount is the amount administered at one time, which may be in the form of more than one administration device (e.g., more than one table, pill or capsule). In some embodiment, the dosage amount is the amount that is administered in a particular period (e.g., one day or one week).

As described herein, any of the pharmaceutical compositions described herein may be administered once, as a single dose. In some embodiments, the pharmaceutical compositions described herein are administered in multiple doses. In some embodiments, each dose is administered in the form of one or more capsules. In some embodiments, each dose comprises administration of multiple capsules. In some embodiments, each dose is administered in the form of 1, 2, 3, 4, 5, 6, 7, 8, 9, 10, 11, 12, 13, 14, 15, or more capsules.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or CFU of each of the bacterial strains per capsule.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacterial cells or CFU per capsule. In some embodiments, each capsule contains between 10⁷ and 10⁹, between 10⁷ and 10⁸, or between 10⁸ and 10⁹ total bacteria. In some embodiments, each capsule contains about 1.0×10⁷, 2.0×10⁷, 3.0×10⁷, 4.0×10⁷, 5.0×10⁷, 6.0×10⁷, 7.0×10⁷, 8.0×10⁷, 9.0×10⁷, 1.0×10⁸, 2.0×10⁸, 3.0×10⁸, 4.0×10⁸, 5.0×10⁸, 6.0×10⁸, 7.0×10⁸, 8.0×10⁸, 9.0×10⁸, 1.0×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2.0×10⁹, 2.1×10⁹, 2.2×10⁹, 2.3×10⁹, 2.4×10⁹, 2.5×10⁹, 2.6×10⁹, 2.7×10⁹, 2.8×10⁹, 2.9×10⁹, 3.0×10⁹, 3.1×10⁹, 3.2×10⁹, 3.3×10⁹, 3.4×10⁹, 3.5×10⁹, 3.6×10⁹, 3.7×10⁹, 3.8×10⁹, 3.9×10⁹, 4.0×10⁹, 4.1×10⁹, 4.2×10⁹, 4.3×10⁹, 4.4×10⁹, 4.5×10⁹, 4.6×10⁹, 4.7×10⁹, 4.8×10⁹, 4.9×10⁹, 5.0×10⁹ total bacterial cells or CFU.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or CFU of each bacterial strain per capsule.

In some embodiments, the pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or CFUs of each of the bacterial strains per dosage amount. In some embodiments, the pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacterial cells or CFUs per dosage amount.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria or colony forming units per milliliter.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of bacteria. In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of bacteria per milliliter.

In some embodiments, the composition includes between 10⁷ and 10⁹, inclusive, bacterial cells or colony forming units of bacteria per milliliter.

In some embodiments, the compositions, including pharmaceutical compositions, contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or CFUs of each of the bacterial strains per dosage amount. In some embodiments, the compositions, including pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacterial cells or CFUs per dosage amount.

In some embodiments, the compositions, including pharmaceutical compositions, contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³, between 10⁻⁶ and 10⁻³, between 10⁻⁵ and 10⁻³, between 10⁻⁴ and 10⁻³, between 10⁻⁷ and 10⁻⁴, between 10⁻⁶ and 10⁻⁴, between 10⁻⁵ and 10⁻⁴, between 10⁻⁷ and 10⁻⁵ between 10⁻⁶ and 10⁻⁵, or between 10⁻⁷ and 10⁻⁶ grams of bacteria of each of the bacterial strains in the composition per dosage amount. In some embodiments, the compositions, including pharmaceutical compositions, disclosed herein contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³, between 10⁻⁶ and 10⁻³, between 10⁻⁵ and 10⁻³, between 10⁻⁴ and 10⁻³, between 10⁻⁷ and 10⁻⁴, between 10⁻⁶ and 10⁻⁴, between 10⁻⁵ and 10⁻⁴, between 10⁻⁷ and 10⁻⁵′ between 10⁻⁶ and 10⁻⁵, or between 10⁻⁷ and 10⁻⁶ grams of all of the bacteria combined (total) per dosage amount.

In some embodiments, if a composition includes more than one bacterial strain, each bacterial strain is present in the composition in the same quantities, in terms of bacterial cells or CFUs. For example, a composition comprising Collinsella aerofaciens and Bifidobacterium adolescentis may comprise 1×10⁸ CFU of Collinsella aerofaciens and 1×10⁸ CFU of Bifidobacterium adolescentis per milliliter, or may comprise 1×10⁸ Collinsella aerofaciens cells and 1×10⁸ Bifidobacterium adolescentis cells per milliliter.

Aspects of the present disclosure also provide food products comprising any of the compositions provided herein and a nutrient. Also within the scope of the present disclosure are food products comprising any of the bacterial strains described herein and a nutrient. Food products are, in general, intended for the consumption of a human or an animal. Any of the bacterial strains described herein may be formulated as a food product. In some embodiments, the bacterial strains are formulated as a food product in spore form. In some embodiments, the bacterial strains are formulated as a food product in vegetative form. In some embodiments, the food product comprises both vegetative bacteria and bacteria in spore form. The compositions disclosed herein can be used in a food or beverage, such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed.

Non-limiting examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

Food products containing bacterial strains described herein may be produced using methods known in the art and may contain the same amount of bacteria (e.g., by weight, amount or CFU) as the pharmaceutical compositions provided herein. Selection of an appropriate amount of bacteria in the food product may depend on various factors, including for example, the serving size of the food product, the frequency of consumption of the food product, the specific bacterial strains contained in the food product, the amount of water in the food product, and/or additional conditions for survival of the bacteria in the food product.

Examples of food products which may be formulated to contain any of the bacterial strains described herein include, without limitation, a beverage, a drink, a bar, a snack, a dairy product, a confectionery product, a cereal product, a ready-to-eat product, a nutritional formula, such as a nutritional supplementary formulation, a food or beverage additive.

### EQUIVALENTS AND SCOPE

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Unless otherwise defined herein, scientific and technical terms used in connection with the present disclosure shall have the meanings that are commonly understood by those of ordinary skill in the art. Further, unless otherwise required by context, singular terms shall include pluralities and plural terms hall include the singular. The methods and techniques of the present disclosure are generally performed according to conventional methods well-known in the art. Generally, nomenclatures used in connection with, and techniques of biochemistry, enzymology, molecular and cellular biology, microbiology, virology, cell or tissue culture, genetics and protein and nucleic chemistry described herein are those well-known and commonly used in the art. The methods and techniques of the present disclosure are generally performed according to conventional methods well known in the art and as described in various general and more specific references that are cited and discussed throughout the present specification unless otherwise indicated.

The present disclosure is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

SEQUENCES
Strain 1
Collinsella aerofaciens 16S ribosomal RNA coding sequence (rDNA)
(SEQ ID NO: 1)
GATTCATTGGGCGTAAAGCGCGCGTAGGCGGCCCGGCAGGCCGGGGGTCGAAGCGGGGGGCTCA
ACCCCCCGAAGCCCCCGGAACCTCCGCGGCTTGGGTCCGGTAGGGGAGGGTGGAACACCCGGTG
TAGCGGTGGAATGCGCAGATATCGGGTGGAACACCGGTGGCGAAGGCGGCCCTCTGGGCCGAGA
CCGACGCTGAGGCGCGAAAGCTGGGGGAGCGAACAGGATTAGATACCCTGGTAGTCCCAGCCGT
AAACGATGGACGCTAGGTGTGGGGGGACGATCCCCCCGTGCCGCAGCCAACGCATTAAGCGTCC
CGCCTGGGGAGTACGGCCGCAAGGCTAAAACTCAAAGGAATTGACGGGGGCCCGCACAAGCAGC
GGAGCATGTGGCTTAATTCGAAGCAACGCGAAGAACCTTACCAGGGCTTGACATATGGGTGAAG
CGGGGGAGACCCCGTGGCCGAGAGGAGCCCATACAGGTGGTGCATGGCTGTCGTCAGCTCGTGT
CGTGAGATGTTGGGTTAAGTCCCGCAACGAGCGCAACCCCCGCCGCGTGTTGCCATCGGGTGAT
GCCGGGAACCCACGCGGGACCGCCGCCGTCAAGGCGGAGGAGGGGGGGGACGACGTCAAGTCAT
CATGCCCCTTATGCCCTGGGCTGCACACGTGCTACAATGGCCGGTACAGAGGGATGCCACCCCG
CGAGGGGGAGCGGATCCCGGAAAGCCGGCCCCAGTTCGGATTGGGGGCTGCAACCCGCCCCCAT
GAAGTCGGAGTTGCTAGTAATCGCGGATCAGCATGCCGCGGTGAATGCGTTCCCGGGCCTTGT
Strain 2
Bifidobacterium adolescentis 16S ribosomal RNA coding sequence
(rDNA)
(SEQ ID NO: 2)
ATTATTGGGCGT-
AAGGGCTCGTAGGCGGTTCGTCGCGTCCGGTGTGAAAGTCCATCGCTTAACGGTGGATCCGCGC
CGGGTACGGGCGGGCTTGAGTGCGGTAGGGGAGACTGGAATTCCCGGTGTAACGGTGGAATGTG
TAGATATCGGGAAGAACACCAATGGCGAAGGCAGGTCTCTGGGCCGTCACTGACGCTGAGGAGC
GAAAGCGTGGGGAGCGAACAGGATTAGATACCCTGGTAGTCCACGCCGTAAACGGTGGATGCTG
GATGTGGGGACCATTCCACGGTCTCCGTGTCGGAGCCAACGCGTTAAGCATCCCGCCTGGGGAG
TACGGCCGCAAGGCTAAAACTCAAAGAAATTGACGGGGGCCCGCACAAGCGGCGGAGCATGCGG
ATTAATTCGATGCAACGCGAAGAACCTTACCTGGGCTTGACATGTTCCCGACAGCCCCAGAGAT
GGGGCCTCCCTTCGGGGGGGGTTCACAGGTGGTGCATGGTCGTCGTCAGCTCGTGTCGTGAGAT
GTTGGGTTAAGTCCCGCAACGAGCGCAACCCTCGCCCTGTGTTGCCAGCACGTCGTGGTGGGAA
CTCACGGGGGACCGCCGGGGTCAACTCGGAGGAAGGTGGGGATGACGTCAGATCATCATGCCCC
TTACGTCCAGGGCTTCACGCATGCTACAATGGCCGGTACAACGGGATGCGACACT-
GTGAGGTGGAGCGGATCCCTTAAAACCGGTCTCAGTTCGGATTGGAGTCTGCAACCCGACTCCA
TGAAGGCGGAGTCGCTAGTAATCGCGGATCAGCAACGCCGCGGTGAATGCGTTCCCGGGCCTTG
TACACACCGCCCGTCAAGTCATGAAAGTGGGTAGCACCCGAAGCCGGTGGCCCAACCTTTTTGG
GGGGAGCCGTCTAAGGTGAGA
Strain 3
Faecalibacteriium prausnitzii 16S ribosomal RNA coding sequence
(rDNA)
(SEQ ID NO: 3)
GCGCG-CT-
ACACATGCAAGTCGAACGAGCGAGAGAGAGCTTGCTTTCTCGAGCGAGTGGCGAACGGGTGAGT
AACGCGTGAGGAACCTGCCTCAAAGAGGGGGACAACAGTTGGAAACGACTGCTAATACCGCATA
AGCCCACGRCYCGGCATCGGGTAGAGGGAAAAGGAGCAATCCGCTTTGAGATGGCCTCGCGTCC
GATTAGCTAGTTGGTGAGGTAAYGGCCCACCAAGGCGACGATCGGTAGCCGGACTGAGAGGTTG
AACGGCCACATTGGGACTGAGACACGGCCCAGACTCCTACGGGAGGCAGCAGTGGGGAATATTG
CACAATGGGGGAAACCCTGATGCAGCGACGCCGCGTGGAGGAAGAAGGTCTTCGGATTGTAAAC
TCCTGTTGTTGAGGAAGATAATGACGGTACTCAACAAGGAAGTGACGGCTAACTACGTGCCAGC
AGCCGCGGTAAAACGTAGGTCACAAGCGTTGTCCGGAATTACTGGGTGTAAAGGGAGCGCAGGC
GGGAAGACAAGTTGGAAGTGAAATCCATGGGCTCAACCCATGAACTGCTTTCAAAACTGTTTTT
CTTGAGTAGTGCAGAGGTAGGCGGAATTCCCGGTGTAGCGGTGGAATGCGTAGATATCGGGAGG
AACACCAGTGGCGAAGGCGGCCTACTGGGCACCAACTGACGCTGAGGCTCGAAAGTGTGGGTAG
CAAACAGGATTAGATACCCTGGTAGTCCACACTGTAAACGATGATTACTAGGTGTTGGAGGATT
GACCCCTTCAGTGCCGCAGTTAACACAATAAGTAATCCACCTGGGGAGTACGACCGCAAGGTTG
AAACTCAAAGGAATTGACGGGGGCCCGCACAAGCAGTGGAGTATGTGGTTTAATTCGACGCAAC
GCGAAGAACCTTACCAAGTCTTGACATCCTGC
Strain 4
Ruminococcus gnavus 16S ribosomal RNA coding sequence (rDNA)
(SEQ ID NO: 4)
CATGCAAGTCGAGCGAAGCACCTTGACGGATTTCTTCGGATTGAAGCCTTGGTGACTGAGCGGC
GGACGGGTGAGTAACGCGTGGGTAACCTGCCTCATACAGGGGGATAACAGTTGGAAACGGCTGC
TAATACCGCATAAGCGCACAGTACCGCATGGTACGGTGTGAAAAACTCCGGTGGTATGAGATGG
ACCCGCGTCTGATTAGGTAGTTGGTGGGGTAACGGCCTACCAAGCCGACGATCAGTAGCCGACC
TGAGAGGGTGACCGGCCACATTGGGACTGAGACACGGCCCAAACTCCTACGGGAGGCAGCAGTG
GGGAATATTGCACAATGGGGGAAACCCTGATGCAGCGACGCCGCGTGAGCGATGAAGTATTTCG
GTATGTAAAGCTCTATCAGCAGGGAAGAAAATGACGGTACCTGACTAAGAAGCCCCGGCTAACT
ACGTGCCAGCAGCCGCGGTAATACGTAGGGGGCAAGCGTTATCCGGATTTACTGGGTGTAAAGG
GAGCGTAGACGGCATGGCAAGCCAGATGTGAAAGCCCGGGGCTCAACCCCGGGACTGCATTTGG
AACTGTCAGGCTAGAGTGTCGGAGAGGAAAGCGGAATTCCTAGTGTAGCGGTGAAATGCGTAGA
TATTAGGAGGAACACCAGTGGCGAAGGCGGCTTTCTGGACGATGACTGACGTTGAGGCTCGAAA
GCGTGGGGAGCAAACAGGATTAGATACCCTGGTAGTCCACGCCGTAAACGATGAATACTAGGTG
KCGGGTGGCAAAGCCATTC
Strain 5
Bacteroides fragilis 16S ribosomal RNA coding sequence (rDNA)
(SEQ ID NO: 5)
GGATTTATTGGGTTT-
AAGGGAGCGTAGGTGGACTGGTAAGTCAGTTGTGAAAGTTTGCGGCTCAACCGTAAAATTGCAG
TTGATACTGTCAGTCTTGAGTACAGTAGAGGTGGGCGGAATTCGTGGTGTAGCGGTGAAATGCT
TAGATATCACGAAGAACTCCGATTGCGAAGGCAGCTCACTGGACTGCAACTGACACTGATGCTC
GAAAGTGTGGGTATCAAACAGGATTAGATACCCTGGTAGTCCACACAGTAAACGATGAATACTC
GCTGTTTGCGATATACAGTAAGCGGCCAAGCGAAAGCATTAAGTATTCCACCTGGGGAGTACGC
CGGCAACGGTGAAACTCAAAGGAATTGACGGGGGCCCGCACAAGCGGAGGAACATGTGGTTTAA
TTCGATGATACGCGAGGAACCTTACCCGGGCTTAAATTGCAGTGGAATGATGTGGAAACATGTC
AGTGAGCAATCACCGCTGTGAAGGTGCTGCATGGTTGTCGTCAGCTCGTGCCGTGAGGTGTCGG
CTTAAGTGCCATAACGAGCGCAACCCTTATCTTTAGTTACTAACAGGTTATGCTGAGGACTCTA
GAGAGACTGCCGTCGTAAGATGTGAGGAAGGTGGGGATGACGTCAAATCAGCACGGCCCTTACG
TCCGGGGCTACACACGTGTTACAATGGGGGGTACAGAAGGCAGCTAGCGGGTGACCGTATGCTA
ATCCCAAAATCCTCTCTCAGTTCGGATCGAAGTCTGCAACCCGACTTCGTGAAGCTGGATTCGC
TAGTAATCGCGCATCAGCCACGGCGCGGTGAATACGTTCCCGGGCCTTGTACACACCGCCCGTC
AAGCCATGGGAGCCGGGGGTACCTGAAGTACGTAACCGCAAGGATCGTCCTAGGGTAAAAC
Strain 6
Anaerostipes caccae 16S ribosomal RNA coding sequence (rDNA)
(SEQ ID NO: 6)
ATTACTGGGTGT-
AAGGGTGCGTAGGTGGCATGGTAAGTCAGAAGTGAAAGCCCGGGGCTTAACCCCGGGACTGCTT
TTGAAACTGTCATGCTGGAGTGCAGGAGAGGTAAGCGGAATTCCTAGTGTAGCGGTGAAATGCG
TAGATATTAGGAGGAACACCAGTGGCGAAGGCGGCTTACTGGACTGTCACTGACACTGATGCAC
GAAAGCGTGGGGAGCAAACAGGATTAGATACCCTGGTAGTCCACGCCGTAAACGATGAATACTA
GGTGTCGGGGCCGTAGAGGCTTCGGTGCCGCAGCAAACGCAGTAAGTATTCCACCTGGGGAGTA
CGTTCGCAAGAATGAAACTCAAAGGAATTGACGGGGACCCGCACAAGCGGTGGAGCATGTGGTT
TAATTCGAAGCAACGCGAAGAACCTTACCTGGTCTTGACATCCCAATGACCGAACCTTAACSGG
WTTTTTCTTTCGAGACAKTKGAGACAGGTGGTGCATGGTTGTCGTCAGCTCGTGTCGTGAGATG
TTGGGTTAAGTCCCGCAACGAGCGCAACCCCTATCTTTAGTAGCCAGCATTTAAGGTGGGCACT
CTAGAGAGACTGCCAGGGATAACCTGGAGGAAGGTGGGGACGACGTCAAATCATCATGCCCCTT
ATGGCCAGGGCTACACACGTGCTACAATGGCGTAAACAAAGGGAAGCGAAGTCGTGAGGCGAAG
CAAATCCCAGAAATAACGTCTCAGTTCGGATTGTAGTCTGCAACTCGACTACATGAAGCTGGAA
TCGCTAGTAATCGTGAATCAGAATGTCACGGTGAATACGTTCCCGGGTCTTGTACACACCGCCC
GTCACACCATGGGAGTCAGTAACGCCCGAAGTCAGTGACCCAACCGCATGGAAGGARCTG
Strain 7
Bacteroides_thetaiotaomicron 16S ribosomal RNA coding sequence
(rDNA)
(SEQ ID NO: 7)
GGATTTATTGGGTTT-
AAGGGAGCGTAGGTGGACAGTTAAGTCAGTTGTGAAAGTTTGCGGCTCAACCGTAAAATTGCAG
TTGATACTGGCTGTCTTGAGTACAGTAGAGGTGGGCGGAATTCGTGGTGTAGCGGTGAAATGCT
TAGATATCACGAAGAACTCCGATTGCGAAGGCAGCTCACTGGACTGCAACTGACACTGATGCTC
GAAAGTGTGGGTATCAAACAGGATTAGATACCCTGGTAGTCCACACAGTAAACGATGAATACTC
GCTGTTTGCGATATACAGTAAGCGGCCAAGCGAAAGCATTAAGTATTCCACCTGGGGAGTACGC
CGGCAACGGTGAAACTCAAAGGAATTGACGGGGGCCCGCACAAGCGGAGGAACATGTGGTTTAA
TTCGATGATACGCGAGGAACCTTACCCGGGCTTAAATTGCATTTGAATATATTGGAAACAGTAT
AGCCGTAAGGCAAATGTGAAGGTGCTGCATGGTTGTCGTCAGCTCGTGCCGTGAGGTGTCGGCT
TAAGTGCCATAACGAGCGCAACCCTTATCTTTAGTTACTAACAGGTCATGCTGAGGACTCTAGA
GAGACTGCCGTCGTAAGATGTGAGGAAGGTGGGGATGACGTCAAATCAGCACGGCCCTTACGTC
CGGGGCTACACACGTGTTACAATGGGGGGTACAGAAGGCAGCTACCTGGTGACAGGATGCTAAT
CCCAAAAGCCTCTCTCAGTTCGGATCGAAGTCTGCAACCCGACTTCGTGAAGCTGGATTCGCTA
GTAATCGCGCATCAGCCATGGCGCGGTGAATACGTTCCCGGGCCTTGTACACACCGCCCGTCAA
GCCATGAAAGCCGGGGGTACCTGAAGTACGTAACCGCAAGGAGCGTCCTAGGGTAAAACT
Strain 8
Bifidobacterium_longum 16S ribosomal RNA coding sequence (rDNA)
(SEQ ID NO: 8)
CGG-ATTATTGGGCGT-
AAGGGCTCGTAGGCGGTTCGTCGCGTCCGGTGTGAAAGTCCATCGCTTAACGGTGGATCCGCGC
CGGGTACGGGCGGGCTTGAGTGCGGTAGGGGAGACTGGAATTCCCGGTGTAACGGTGGAATGTG
TAGATATCGGGAAGAACACCAATGGCGAAGGCAGGTCTCTGGGCCGTTACTGACGCTGAGGAGC
GAAAGCGTGGGGAGCGAACAGGATTAGATACCCTGGTAGTCCACGCCGTAAACGGTGGATGCTG
GATGTGGGGCCCGTTCCACGGGTTCCGTGTCGGAGCTAACGCGTTAAGCATCCCGCCTGGGGAG
TACGGCCGCAAGGCTAAAACTCAAAGAAATTGACGGGGGCCCGCACAAGCGGCGGAGCATGCGG
ATTAATTCGATGCAACGCGAAGAACCTTACCTGGGCTTGACATGTTCCCGACGATCCCAGAGAT
GGGGTTTCCCTTCGGGGGGGGTTCACAGGTGGTGCATGGTCGTCGTCAGCTCGTGTCGTGAGAT
GTTGGGTTAAGTCCCGCAACGAGCGCAACCCTCGCCCCGTGTTGCCAGCGGATTGTGCCGGGAA
CTCACGGGGGACCGCCGGGGTTAACTCGGAGGAAGGTGGGGATGACGTCAGATCATCATGCCCC
TTACGTCCAGGGCTTCACGCATGCTACAATGGCCGGTACAACGGGATGCGACGCGGCGACGCGG
AGCGGATCCCTGAAAACCGGTCTCAGTTCGGATCGCAGTCTGCAACTCGACTGCGTGAAGGCGG
AGTCGCTAGTAATCGCGAATCAGCAACGTCGCGGTGAATGCGTTCCCGGGCCTTGTACACACCG
CCCGTCAAGTCATGAAAGTGGGCAGCACCCGAAGCCGGTGGCCTAACCCCTTGTGGGATGGAGC
CGTCTAAGGTGAGGCTCGTG
Strain 9
Akkermansia_muciniphila 16S ribosomal RNA coding sequence (rDNA)
(SEQ ID NO: 9)
CGATGTTCGG-ATCACTGGGCGT-
AAGCGTGCGTAGGCGGTTTCGTAAGTCGTGTGTGAAAGGCGGGGGCTCAACCCCCGGACTGCAC
ATGATACTGCGAGACTAGAGTAATGGAGGGGGAACCGGAATTCTCGGTGTAGCAGTGAAATGCG
TAGATATCGAGAGGAACACTCGTGGCGAAGGCGGGTTCCTGGACATTAACTGACGCTGAGGCAC
GAAGGCCAGGGGAGCGAAAGGGATTAGATACCCCTGTAGTCCTGGCAGTAAACGGTGCACGCTT
GGTGTGCGGGGAATCGACCCCCTGCGTGCCGGAGCTAACGCGTTAAGCGTGCCGCCTGGGGAGT
ACGGTCGCAAGATTAAAACTCAAAGAAATTGACGGGGACCCGCACAAGCGGTGGAGTATGTGGC
TTAATTCGATGCAACGCGAAGAACCTTACCTGGGCTTGACATGTAATGAACAACATGTGAAAGC
ATGCGACTCTTCGGAGGCGTTACACAGGTGCTGCATGGCCGTCGTCAGCTCGTGTCGTGAGATG
TTTGGTTAAGTCCAGCAACGAGCGCAACCCCTGTTGCCAGTTACCAGCACGTAAAGGTGGGGAC
TCTGGCGAGACTGCCCAGATCAACTGGGAGGAAGGTGGGGACGACGTCAGGTCAGTATGGCCCT
TATGCCCAGGGCTGCACACGTACTACAATGCCCAGTACAGAGGGGGCCGAAGCCGCGAGGCGGA
GGAAATCCTAAAAACTGGGCCCAGTTCGGACTGTAGGCTGCAACCCGCCTACACGAAGCCGGAA
TCGCTAGTAATGGCGCATCAGCTACGGCGCCGTGAATACGTTCCCGGGTCTTGTACACACCGCC
CGTCACATCATGGAAGCCGGTCGCACCCGAAGTAT
Strain 10
Fusobacterium ulcerans 16S ribosomal RNA coding sequence (rDNA)
(SEQ ID NO: 10)
CGAAGAGTTTGATCCTGGCTCAGGATGAACGCTGACAGAATGCTTAACACATGCAAGTCTACTT
GATCCTTCGGGTGAAGGTGGCGGACGGGTGAGTAACGCGTAAAGAACTTGCCTTACAGACTGGG
ACAACATTTGGAAACGAATGCTAATACCGGATATTATGATTGGGTCGCATGATCTGATTATGAA
AGCTATATGCGCTGTGAGAGAGCTTTGCGTCCCATTAGTTAGTTGGTGAGGTAACGGCTCACCA
AGACGATGATGGGTAGCCGGCCTGAGAGGGTGAACGGCCACAAGGGGACTGAGACACGGCCCTT
ACTCCTACGGGAGGCAGCAGTGGGGAATATTGGACAATGGACCAAAAGTCTGATCCAGCAATTC
TGTGTGCACGAAGAAGTTTTTCGGAATGTAAAGTGCTTTCAGTTGGGAAGAAGTCAGTGACGGT
ACCAACAGAAGAAGCGACGGCTAAATACGTGCCAGCAGCCGCGGTAATACGTATGTCGCAAGCG
TTATCCGGATTTATTGGGCGTAAAGCGCGTCTAGGCGGCTTAGTAAGTCTGATGTGAAAATGCG
GGGCTCAACCCCGTATTGCGTTGGAAACTGCTAAACTAGAGTACTGGAGAGGTAGGCGGAACTA
CAAGTGTAGAGGTGAAATTCGTAGATATTTGTAGGAATGCCGATGGGGAAGCCAGCCTACTGGA
CAGATACTGACGCTAAAGCGCGAAAGCGTGGGTAGCAAACAGGATTAGATACCCTGGTAGTCCA
CGCCGTAAACGATGATTACTAGGTGTTGGGGGTCGAACCTCAGCGCCCAAGCTAACGCGATAAG
TAATCCGCCTGGGGAGTACGTACGCAAGTATGAAACTCAAAGGAATTGACGGGGACCCGCACAA
GCGGTGGAGCATGTGGTTTAATTCGACGCAACGCGAGGAACCTTACCAGCGTTTGACATCCCAA
GAAGTTAACAGAGATGTTTTCGTGCCTCTTCGGAGGAACTTGGTGACAGGTGGTGCATGGCTGT
CGTCAGCTCGTGTCGTGAGATGTTGGGTTAAGTCCCGCAACGAGCGCAACCCCTTTCGTATGTT
ACCATCATTAAGTTGGGGACTCATGCGAGACTGCCTGCGATGAGCAGGAGGAAGGTGGGGATGA
CGTCAAGTCATCATGCCCCTTATACGCTGGGCTACACACGTGCTACAATGGGTAGTACAGAGAG
CTGCAAACCTGCGAGGGTAAGCTAATCTCATAAAACTATTCTTAGTTCGGATTGTACTCTGCAA
CTCGAGTACATGAAGTTGGAATCGCTAGTAATCGCAAATCAGCTATGTTGCGGTGAATACGTTC
TCGGGTCTTGTACACACCGCCCGTCACACCACGAGAGTTGGTTGCACCTGAAGTAACAGGCCTA
ACCGTAAGGAGGGATGTTCCGAGGGTGTGATTAGCGATTGGGGTGAAGTCGTAACAAGGTATCC
GTACGGGAACGTGCGGATGGATCACCTCCTT
Strain 11
Eubacterium limosum 16S ribosomal RNA coding sequence (rDNA)
(SEQ ID NO: 11)
TATTGAGAGTTTGATCCTGGCTCAGGACGAACGCTGGCGGTATGCTTAACACATGCAAGTCGAA
CGAGAAGGTTTTGATGGATCCTTCGGGTGATATCAGAACTGGAAAGTGGCGAACGGGTGAGTAA
CGCGTGGGTAACCTGCCCTATGGAAAGGAATAGCCTCGGGAAACTGGGAGTAAAGCCTTATATT
ATGGTTTTGTCGCATGGCAAGATCATGAAAACTCCGGTGCCATAGGATGGACCCGCGTCCCATT
AGCTAGTTGGTGAGATAACAGCCCACCAAGGCGACGATGGGTAACCGGTCTGAGAGGGCGAACG
GTCACACTGGAACTGAGACACGGTCCAGACTCCTACGGGAGGCAGCAGTGGGGAATATTGCGCA
ATGGGGGCAACCCTGACGCAGCAATACCGCGTGAGTGAAGAAGGTTTTCGGATCGTAAAGCTCT
GTTATTGGGGAAGAAGAATGACGGTACCCAATGAGGAAGTCCCGGCTAACTACGTGCCAGCAGC
CGCGGTAATACGTAGGGGACAAGCGTTGTCCGGAATGACTGGGCGTAAAGGGCGCGTAGGCGGT
CTATTAAGTCTGATGTGAAAGGTACCGGCTCAACCGGTGAAGTGCATTGGAAACTGGTAGACTT
GAGTATTGGAGAGGCAAGTGGAATTCCTAGTGTAGCGGTGAAATGCGTAGATATTAGGAGGAAC
ACCAGTGGCGAAGGCGGCTTGCTGGACAAATACTGACGCTGAGGTGCGAAAGCGTGGGGAGCGA
ACAGGATTAGATACCCTGGTAGTCCACGCCGTAAACGATGAATGCTAGGTGTTGGGGAAACTCA
GTGCCGCAGTTAACACAATAAGCATTCCGCCTGGGGAGTACGACCGCAAGGTTGAAACTCAAAG
GAATTGACGGGGACCCGCACAAGCAGCGGAGCATGTGGTTTAATTCGAAGCAACGCGAAGAACC
TTACCAGGTCTTGACATCCTCTGACGAGCCTAGAGATAGGAAGTTTCCTTCGGGAACAGAGAGA
CAGGTGGTGCATGGTTGTCGTCAGCTCGTGTCGTGAGATGTTGGGTTAAGTCCCGCAACGAGCG
CAACCCCTGCCTTTAGTTGCCAGCATTAAGTTGGGCACTCTAGAGGGACTGCCGTAGACAATAC
GGAGGAAGGTGGGGACGACGTCAAATCATCATGCCCCTTATGACCTGGGCTACACACGTGCTAC
AATGGTCTGAACAGAGGGCCGCGAAGCCGCGAGGTGAAGCAAATCCCTTAAAACAGATCCCAGT
TCGGATTGCAGGCTGCAACTCGCCTGCATGAAGTTGGAGTTGCTAGTAATCGCGGATCAGAATG
CCGCGGTGAATGCGTTCCCGGGTCTTGTACACACCGCCCGTCACACCACGAGAGTTGGCAACAC
CCGAAGCCTGTGAGAGAACCGTAAGGACTCAGCAGTCGAAGGTGGGGCTAGTAATTGGGGTGAA
GTCGTAACAAGGTAGCCGTATCGGAAGGTGCGGCTGGATCACCTCCTTT
Strain 12
Subdoligranulum sp. 16S ribosomal RNA coding sequence (rDNA)
(SEQ ID NO: 12)
AATGAAGAGTTTGATCCTGGCTCAGGACGAACGCTGGCGGCGCGCCTAACACATGCAAGTCGAA
CGGAGCTGTTTTCTCTGAAGTTTTCGGATGGAAGAGAGTTCAGCTTAGTGGCGAACGGGTGAGT
AACACGTGAGCAACCTGCCTTTCAGTGGGGGACAACATTTGGAAACGAATGCTAATACCGCATA
AGACCACAGTGTCGCATGGCACAGGGGTCAAAGGATTTATCCGCTGAAAGATGGGCTCGCGTCC
GATTAGCTAGATGGTGAGGTAACGGCCCACCATGGCGACGATCGGTAGCCGGACTGAGAGGTTG
AACGGCCACATTGGGACTGAGACACGGCCCAGACTCCTACGGGAGGCAGCAGTGGGGAATATTG
CACAATGGGGGAAACCCTGATGCAGCGACGCCGCGTGGAGGAAGAAGGTCTTCGGATTGTAAAC
TCCTGTCCCAGGGGACGATAATGACGGTACCCTGGGAGGAAGCACCGGCTAACTACGTGCCAGC
AGCCGCGGTAAAACGTAGGGTGCAAGCGTTGTCCGGAATTACTGGGTGTAAAGGGAGCGCAGGC
GGATTGGCAAGTTGGGAGTGAAATCTATGGGCTCAACCCATAAATTGCTTTCAAAACTGTCAGT
CTTGAGTGGTGTAGAGGTAGGCGGAATTCCCGGTGTAGCGGTGGAATGCGTAGATATCGGGAGG
AACACCAGTGGCGAAGGCGGCCTACTGGGCACTAACTGACGCTGAGGCTCGAAAGCATGGGTAG
CAAACAGGATTAGATACCCTGGTAGTCCATGCCGTAAACGATGATTACTAGGTGTGGGAGGATT
GACCCCTTCCGTGCCGCAGTTAACACAATAAGTAATCCACCTGGGGAGTACGACCGCAAGGTTG
AAACTCAAAGGAATTGACGGGGGCCCGCACAAGCAGTGGAGTATGTGGTTTAATTCGAAGCAAC
GCGAAGAACCTTACCAGGTCTTGACATCGGATGCATACCTAAGAGATTAGGGAAGTCCTTCGGG
ACATCCAGACAGGTGGTGCATGGTTGTCGTCAGCTCGTGTCGTGAGATGTTGGGTTAAGTCCCG
CAACGAGCGCAACCCTTATCGTTAGTTACTACGCAAGAGGACTCTAGCGAGACTGCCGTTGACA
AAACGGAGGAAGGTGGGGATGACGTCAAATCATCATGCCCTTTATGACCTGGGCTACACACGTA
CTACAATGGCTATTAACAGAGAGAAGCGATACCGCGAGGTGGAGCAAACCTCACAAAAATAGTC
TCAGTTCGGATCGCAGGCTGCAACCCGCCTGCGTGAAGCCGGAATTGCTAGTAATCGCGGATCA
GCATGCCGCGGTGAATACGTTCCCGGGCCTTGTACACACCGCCCGTCACACCATGAGAGCCGGG
GGGACCCGAAGTCGGTAGTCTAACCGCAAGGAGGACGCCGCCGAAGGTAAAACTGGTGATTGGG
GTGAAGTCGTAACAAGGTAGCCGTATCGGAAGGTGCGGCTGGATCACCTCCTTT
Strain 13
Phascolarctobacterium faecium 16S ribosomal RNA coding sequence
(rDNA)
(SEQ ID NO: 13)
ATTGGAGAGTTTGATCCTGGCTCAGGACGAACGCTGGCGGCGTGCCTAACACATGCAAGTCGAA
CGGAGAATTTTATTTCGGTAGAATTCTTAGTGGCGAACGGGTGAGTAACGCGTAGGCAACCTAC
CCTTTAGACGGGGACAACATTCCGAAAGGAGTGCTAATACCGGATGTGATCATCTTGCCGCATG
GCAGGATGAAGAAAGATGGCCTCTACAAGTAAGCTATCGCTAAAGGATGGGCCTGCGTCTGATT
AGCTAGTTGGTAGTGTAACGGACTACCAAGGCGATGATCAGTAGCCGGTCTGAGAGGATGAACG
GCCACATTGGGACTGAGACACGGCCCAAACTCCTACGGGAGGCAGCAGTGGGGAATCTTCCGCA
ATGGACGAAAGTCTGACGGAGCAACGCCGCGTGAGTGATGAAGGATTTCGGTCTGTAAAGCTCT
GTTGTTTATGACGAACGTGCAGTGTGTGAACAATGCATTGCAATGACGGTAGTAAACGAGGAAG
CCACGGCTAACTACGTGCCAGCAGCCGCGGTAATACGTAGGTGGCGAGCGTTGTCCGGAATTAT
TGGGCGTAAAGAGCATGTAGGCGGCTTAATAAGTCGAGCGTGAAAATGCGGGGCTCAACCCCGT
ATGGCGCTGGAAACTGTTAGGCTTGAGTGCAGGAGAGGAAAGGGGAATTCCCAGTGTAGCGGTG
AAATGCGTAGATATTGGGAGGAACACCAGTGGCGAAGGCGCCTTTCTGGACTGTGTCTGACGCT
GAGATGCGAAAGCCAGGGTAGCGAACGGGATTAGATACCCCGGTAGTCCTGGCCGTAAACGATG
GGTACTAGGTGTAGGAGGTATCGACCCCTTCTGTGCCGGAGTTAACGCAATAAGTACCCCGCCT
GGGGAGTACGGCCGCAAGGTTGAAACTCAAAGGAATTGACGGGGGCCCGCACAAGCGGTGGAGT
ATGTGGTTTAATTCGACGCAACGCGAAGAACCTTACCAAGGCTTGACATTGATTGAACGCTCTA
GAGATAGAGATTTCCCTTCGGGGACAAGAAAACAGGTGGTGCATGGCTGTCGTCAGCTCGTGTC
GTGAGATGTTGGGTTAAGTCCCGCAACGAGCGCAACCCCTATCCTATGTTACCAGCAAGTAAAG
TTGGGGACTCATGGGAGACTGCCAGGGACAACCTGGAGGAAGGCGGGGATGACGTCAAGTCATC
ATGCCCCTTATGTCTTGGGCTACACACGTACTACAATGGTCGGAAACAGAGGGAAGCGAAGCCG
CGAGGCAGAGCAAACCCCAGAAACCCGATCTCAGTTCGGATCGCAGGCTGCAACCCGCCTGCGT
GAAGTCGGAATCGCTAGTAATCGCAGGTCAGCATACTGCGGTGAATACGTTCCCGGGCCTTGTA
CACACCGCCCGTCACACCACGAAAGTTGGTAACACCCGAAGCCGGTGAGGTAACCTATTAGGAG
CCAGCCGTCTAAGGTGGGGCCGATGATTGGGGTGAAGTCGTAACAAGGTAGCCGTATCGGAAGG
TGCGGCTGGATCACCTCCTTT
Strain 14
Parabacteroides johnsonii 16S ribosomal RNA coding sequence
(rDNA)
(SEQ ID NO: 14)
CGAAGAGTTTGATCCTGGCTCAGGATGAACGCTAGCGACAGGCTTAACACATGCAAGTCGAGGG
GCATCATGGTAAGTAGCAATACTTATTGATGGCGACCGGCGCACGGGTGAGTAACGCGTATGCA
ACTTACCTATCAGAGGGGGATAGCCCGGCGAAAGTCGGATTAATACTCCATAAAACAGGGGTTC
CGCATGGGACTATTTGTTAAAGATTCATCGCTGATAGATAGGCATGCGTTCCATTAGGCAGTTG
GCGGGGTAACGGCCCACCAAACCGACGATGGATAGGGGTTCTGAGAGGAAGGTCCCCCACATTG
GTACTGAGACACGGACCAAACTCCTACGGGAGGCAGCAGTGAGGAATATTGGTCAATGGCCGAG
AGGCTGAACCAGCCAAGTCGCGTGAAGGATGAAGGATCTATGGTTTGTAAACTTCTTTTATAGG
GGAATAAAGTGTGGGACGTGTTCCATTTTGTATGTACCCTATGAATAAGCATCGGCTAACTCCG
TGCCAGCAGCCGCGGTAATACGGAGGATGCGAGCGTTATCCGGATTTATTGGGTTTAAAGGGTG
CGTAGGTGGTAATTTAAGTCAGCGGTGAAAGTTTGTGGCTCAACCATAAAATTGCCGTTGAAAC
TGGGTTACTTGAGTGTGTTTGAGGTAGGCGGAATGCGTGGTGTAGCGGTGAAATGCATAGATAT
CACGCAGAACTCCAATTGCGAAGGCAGCTTACTAAACCATAACTGACACTGAAGCACGAAAGCG
TGGGTATCAAACAGGATTAGATACCCTGGTAGTCCACGCAGTAAACGATGATTACTAGGAGTTT
GCGATACACAGTAAGCTCTACAGCGAAAGCGTTAAGTAATCCACCTGGGGAGTACGCCGGCAAC
GGTGAAACTCAAAGGAATTGACGGGGGCCCGCACAAGCGGAGGAACATGTGGTTTAATTCGATG
ATACGCGAGGAACCTTACCCGGGTTTGAACGTAGTCAGACCGACCTTGAAAGAGGTTTTCTAGC
AATAGCTGATTACGAGGTGCTGCATGGTTGTCGTCAGCTCGTGCCGTGAGGTGTCGGCTTAAGT
GCCATAACGAGCGCAACCCTTATCACTAGTTACTAACAGGTTAAGCTGAGGACTCTGGTGAGAC
TGCCAGCGTAAGCTGTGAGGAAGGTGGGGATGACGTCAAATCAGCACGGCCCTTACATCCGGGG
CGACACACGTGTTACAATGGCATGGACAAAGGGCAGCTACCTGGCGACAGGATGCTAATCTCTA
AACCATGTCTCAGTTCGGATCGGAGTCTGCAACTCGACTCCGTGAAGCTGGATTCGCTAGTAAT
CGCGCATCAGCCATGGCGCGGTGAATACGTTCCCGGGCCTTGTACACACCGCCCGTCAAGCCAT
GGGAGCCGGGGGTACCTGAAGTCCGTAACCGCAAGGATCGGCCTAGGGTAAAACTGGTGACTGG
GGCTAAGTCGTAACAAGGTAGCCGTACCGGAAGGTGCGGCTGGAACACCTCCTT
Strain 15
Paraprevotella xylaniphila 16S ribosomal RNA coding sequence
(rDNA)
(SEQ ID NO: 15)
AATAAAGATTAATTGGTAAAGGATGGGGATGCGTCCCATTAGCTTGTTGGCGGGGTAACGGCCC
ACCAAGGCGACGATGGGTAGGGGTTCTGAGAGGAAGGTCCCCCACATTGGAACTGAGACACGGT
CCAAACTCCTACGGGAGGCAGCAGTGAGGAATATTGGTCAATGGGCGCGAGCCTGAACCAGCCA
AGTAGCGTGGAGGACGACGGCCCTACGGGTTGTAAACTCCTTTTATAAGGGGATAAAGTTGGCC
ATGTATGGCCATTTGCAGGTACCTTATGAATAAGCATCGGCTAATTCCGTGCCAGCAGCCGCGG
TAATACGGAAGATGCGAGCGTTATCCGGATTTATTGGGTTTAAAGGGAGCGTAGGCGGGCAGTC
AAGTCAGCGGTCAAATGGCGCGGCTCAACCGCGTTCCGCCGTTGAAACTGGCAGCCTTGAGTAT
GCACAGGGTACATGGAATTCGTGGTGTAGCGGTGAAATGCTTAGATATCACGAGGAACTCCGAT
CGCGCAGGCATTGTACCGGGGCATTACTGACGCTGAGGCTCGAAGGTGCGGGTATCAAACAGGA
TTAGATACCCTGGTAGTCCGCACAGTAAACGATGAATGCCCGCTGTCGGCGACATAGTGTCGGC
GGCCAAGCGAAAGCGTTAAGCATTCCACCTGGGGAGTACGCCGGCAACGGTGAAACTCAAAGGA
ATTGACGGGGGCCCGCACAAGCGGAGGAACATGTGGTTTAATTCGATGATACGCGAGGAACCTT
ACCCGGGCTTGAATCGCAGGTGCATGGGCCGGAGACGGCCCTTTCCTTCGGGACTCCTGCGAAG
GTGCTGCATGGTTGTCGTCAGCTCGTGCCGTGAGGTGTCGGCTTAAGTGCCATAACGAGCGCAA
CCCCCCTCCCCAGTTGCCACCGGGTAATGCCGGGCACTTTGGGGACACTGCCACCGCAAGGTGC
GAGGAAGGTGGGGATGACGTCAAATCAGCACGGCCCTTACGTCCGGGGCGACACACGTGTTACA
ATGGGGGGTACAGAGGGCCGCTGCCCGGTGACGGTTGGCCAATCCCTAAAACCCCTCTCAGTTC
GGACTGGAGTCTGCAACCCGACTCCACGAAGCTGGATTCGCTAGTAATCGCGCATCAGCCATGG
CGCGGTGAATACGTTCCCGGGCCTTGTACACACCGCCCGTCAAGCCATGAAAGCCGGGGGTGCC
TGAAGTCCGTGACCGCGAGGGTCGGCCTAGGGTAAAACCGGTGATTGGGGCTAAGTCGTAACAA
GGTAGCCGTACCGGAAGGTGCGGCTGGAACACCTCCTTT
Strain 16
Parabacteroides distasonis 16S ribosomal RNA coding sequence
(rDNA)
(SEQ ID NO: 16)
CGAAGAGTTTGATCCTGGCTCAGGATGAACGCTAGCGACAGGCTTAACACATGCAAGTCGAGGG
GCAGCACAGGTAGCAATACCGGGTGGCGACCGGCGCACGGGTGAGTAACGCGTATGCAACTTGC
CTATCAGAGGGGGATAACCCGGCGAAAGTCGGACTAATACCGCATGAAGCAGGGGCCCCGCATG
GGGATATTTGCTAAAGATTCATCGCTGATAGATAGGCATGCGTTCCATTAGGCAGTTGGCGGGG
TAACGGCCCACCAAACCGACGATGGATAGGGGTTCTGAGAGGAAGGTCCCCCACATTGGTACTG
AGACACGGACCAAACTCCTACGGGAGGCAGCAGTGAGGAATATTGGTCAATGGCCGAGAGGCTG
AACCAGCCAAGTCGCGTGAGGGATGAAGGTTCTATGGATCGTAAACCTCTTTTATAAGGGAATA
AAGTGCGGGACGTGTCCCGTTTTGTATGTACCTTATGAATAAGGATCGGCTAACTCCGTGCCAG
CAGCCGCGGTAATACGGAGGATCCGAGCGTTATCCGGATTTATTGGGTTTAAAGGGTGCGTAGG
CGGCCTTTTAAGTCAGCGGTGAAAGTCTGTGGCTCAACCATAGAATTGCCGTTGAAACTGGGGG
GCTTGAGTATGTTTGAGGCAGGCGGAATGCGTGGTGTAGCGGTGAAATGCATAGATATCACGCA
GAACCCCGATTGCGAAGGCAGCCTGCCAAGCCATTACTGACGCTGATGCACGAAAGCGTGGGGA
TCAAACAGGATTAGATACCCTGGTAGTCCACGCAGTAAACGATGATCACTAGCTGTTTGCGATA
CACTGTAAGCGGCACAGCGAAAGCGTTAAGTGATCCACCTGGGGAGTACGCCGGCAACGGTGAA
ACTCAAAGGAATTGACGGGGGCCCGCACAAGCGGAGGAACATGTGGTTTAATTCGATGATACGC
GAGGAACCTTACCCGGGTTTGAACGCATTCGGACCGAGGTGGAAACACCTTTTCTAGCAATAGC
CGTTTGCGAGGTGCTGCATGGTTGTCGTCAGCTCGTGCCGTGAGGTGTCGGCTTAAGTGCCATA
ACGAGCGCAACCCTTGCCACTAGTTACTAACAGGTAAAGCTGAGGACTCTGGTGGGACTGCCAG
CGTAAGCTGCGAGGAAGGCGGGGATGACGTCAAATCAGCACGGCCCTTACATCCGGGGCGACAC
ACGTGTTACAATGGCGTGGACAAAGGGAAGCCACCTGGCGACAGGGAGCGAATCCCCAAACCAC
GTCTCAGTTCGGATCGGAGTCTGCAACCCGACTCCGTGAAGCTGGATTCGCTAGTAATCGCGCA
TCAGCCATGGCGCGGTGAATACGTTCCCGGGCCTTGTACACACCGCCCGTCAAGCCATGGGAGC
CGGGGGTACCTGAAGTCCGTAACCGCGAGGATCGGCCTAGGGTAAAACTGGTGACTGGGGCTAA
GTCGTAACAAGGTAGCCGTACCGGAAGGTGCGGCTGGAACACCTCCTTT
Strain 17
Alistipes sp. 16S ribosomal RNA coding sequence (rDNA)
(SEQ ID NO: 17)
ATGGAGAGTTTGATCCTGGCTCAGGATGAACGCTAGCGGCAGGCCTAACACATGCAAGTCGAGG
GGCAGCGGGATTGAAGCTTGCTTCAGTTGCCGGCGACCGGCGCACGGGTGCGTAACGCGTATGC
AACCTACCCATAACAGGGGGATAACACTGAGAAATCGGTACTAATATCCCATAACATCAAGAGG
GGCATCCCTTTTGGTTGAAAACTCCGGTGGTTATGGATGGGCATGCGTTGTATTAGCTAGTTGG
TGAGGTAACGGCTCACCAAGGCGACGATACATAGGGGGACTGAGAGGTTAACCCCCCACATTGG
TACTGAGACACGGACCAAACTCCTACGGGAGGCAGCAGTGAGGAATATTGGTCAATGGACGCAA
GTCTGAACCAGCCATGCCGCGTGCAGGATGACGGCTCTATGAGTTGTAAACTGCTTTTGTACGA
GGGTAAACCCGGATACGTGTATCCGGCTGAAAGTATCGTACGAATAAGGATCGGCTAACTCCGT
GCCAGCAGCCGCGGTAATACGGAGGATTCAAGCGTTATCCGGATTTATTGGGTTTAAAGGGTGC
GTAGGCGGTTTGATAAGTTAGAGGTGAAATACCGGTGCTTAACACCGGAACTGCCTCTAATACT
GTTGAGCTAGAGAGTAGTTGCGGTAGGCGGAATGTATGGTGTAGCGGTGAAATGCTTAGAGATC
ATACAGAACACCGATTGCGAAGGCAGCTTACCAAACTATATCTGACGTTGAGGCACGAAAGCGT
GGGGAGCAAACAGGATTAGATACCCTGGTAGTCCACGCAGTAAACGATGATAACTCGCTGTCGG
CGATACACAGTCGGTGGCTAAGCGAAAGCGATAAGTTATCCACCTGGGGAGTACGTTCGCAAGA
ATGAAACTCAAAGGAATTGACGGGGGCCCGCACAAGCGGAGGAACATGTGGTTTAATTCGATGA
TACGCGAGGAACCTTACCCGGGCTTGAAAGTTACTGACGATTCTGGAAACAGGATTTCCCTTCG
GGGCAGGAAACTAGGTGCTGCATGGTTGTCGTCAGCTCGTGCCGTGAGGTGTCGGGTTAAGTCC
CATAACGAGCGCAACCCCTACCGTTAGTTGCCATCAGGTCAAGCTGGGCACTCTGGCGGGACTG
CCGGTGTAAGCCGAGAGGAAGGTGGGGATGACGTCAAATCAGCACGGCCCTTACGTCCGGGGCT
ACACACGTGTTACAATGGTAGGTACAGAGGGCAGCTACCCAGTGATGGGATGCGAATCTCGAAA
GCCTATCTCAGTTCGGATTGGAGGCTGAAACCCGCCTCCATGAAGTTGGATTCGCTAGTAATCG
CGCATCAGCCATGGCGCGGTGAATACGTTCCCGGGCCTTGTACACACCGCCCGTCAAGCCATGG
AAGCTGGGGGTGCCTGAAGTTCGTGACCGCAAGGAGCGACCTAGGGCAAAACCGGTGACTGGGG
CTAAGTCGTAACAAGGTAGCCGTACCGGAAGGTGCGGCTGGAACACCTCCTTT
Strain 18
Bacteroides dorei 16S ribosomal RNA coding sequence (rDNA)
(SEQ ID NO: 18)
ATGAAGAGTTTGATCCTGGCTCAGGATGAACGCTAGCTACAGGCTTAACACATGCAAGTCGAGG
GGCAGCATGGTCTTAGCTTGCTAAGGCTGATGGCGACCGGCGCACGGGTGAGTAACACGTATCC
AACCTGCCGTCTACTCTTGGCCAGCCTTCTGAAAGGAAGATTAATCCAGGATGGGATCATGAGT
TCACATGTCCGCATGATTAAAGGTATTTTCCGGTAGACGATGGGGATGCGTTCCATTAGATAGT
AGGCGGGGTAACGGCCCACCTAGTCAACGATGGATAGGGGTTCTGAGAGGAAGGTCCCCCACAT
TGGAACTGAGACACGGTCCAAACTCCTACGGGAGGCAGCAGTGAGGAATATTGGTCAATGGGCG
ATGGCCTGAACCAGCCAAGTAGCGTGAAGGATGACTGCCCTATGGGTTGTAAACTTCTTTTATA
AAGGAATAAAGTCGGGTATGCATACCCGTTTGCATGTACTTTATGAATAAGGATCGGCTAACTC
CGTGCCAGCAGCCGCGGTAATACGGAGGATCCGAGCGTTATCCGGATTTATTGGGTTTAAAGGG
AGCGTAGATGGATGTTTAAGTCAGTTGTGAAAGTTTGCGGCTCAACCGTAAAATTGCAGTTGAT
ACTGGATGTCTTGAGTGCAGTTGAGGCAGGCGGAATTCGTGGTGTAGCGGTGAAATGCTTAGAT
ATCACGAAGAACTCCGATTGCGAAGGCAGCCTGCTAAGCTGCAACTGACATTGAGGCTCGAAAG
TGTGGGTATCAAACAGGATTAGATACCCTGGTAGTCCACACGGTAAACGATGAATACTCGCTGT
TTGCGATATACGGCAAGCGGCCAAGCGAAAGCGTTAAGTATTCCACCTGGGGAGTACGCCGGCA
ACGGTGAAACTCAAAGGAATTGACGGGGGCCCGCACAAGCGGAGGAACATGTGGTTTAATTCGA
TGATACGCGAGGAACCTTACCCGGGCTTAAATTGCACTCGAATGATCCGGAAACGGTTCAGCTA
GCAATAGCGAGTGTGAAGGTGCTGCATGGTTGTCGTCAGCTCGTGCCGTGAGGTGTCGGCTTAA
GTGCCATAACGAGCGCAACCCTTGTTGTCAGTTACTAACAGGTGATGCTGAGGACTCTGACAAG
ACTGCCATCGTAAGATGTGAGGAAGGTGGGGATGACGTCAAATCAGCACGGCCCTTACGTCCGG
GGCTACACACGTGTTACAATGGGGGGTACAGAGGGCCGCTACCACGCGAGTGGATGCCAATCCC
TAAAACCCCTCTCAGTTCGGACTGGAGTCTGCAACCCGACTCCACGAAGCTGGATTCGCTAGTA
ATCGCGCATCAGCCACGGCGCGGTGAATACGTTCCCGGGCCTTGTACACACCGCCCGTCAAGCC
ATGGGAGCCGGGGGTACCTGAAGTGCGTAACCGCGAGGATCGCCCTAGGGTAAAACTGGTGACT
GGGGCTAAGTCGTAACAAGGTAGCCGTACCGGAAGGTGCGGCTGGAACACCTCCTT
Strain 19
Parabacteroides gordonii 16S ribosomal RNA coding sequence
(rDNA)
(SEQ ID NO: 19)
CGAAGAGTTTGATCCTGGCTCAGGATGAACGCTAGCGACAGGCTTAACACATGCAAGTCGAGGG
GCAGCAGGAAGTAGCAATACTTTGCTGGCGACCGGCGCACGGGTGAGTAACGCGTATGCAACCT
ACCTATCAGAGGGGGATAACCCGGCGAAAGTCGGACTAATACCGCATAAAACAGGGGTCCCGCA
TGGGAATATTTGTTAAAGATTTATTGCTGATAGATGGGCATGCGTTCCATTAGATAGTTGGTGA
GGTAACGGCTCACCAAGTCTTCGATGGATAGGGGTTCTGAGAGGAAGGTCCCCCACACTGGTAC
TGAGACACGGACCAGACTCCTACGGGAGGCAGCAGTGAGGAATATTGGTCAATGGGCGAGAGCC
TGAACCAGCCAAGTCGCGTGAAGGATGAAGGATCTATGGTTCGTAAACTTCTTTTATAGGGGAA
TAAAGTGCAGGACGTGTCCTGTTTTGTATGTACCCTATGAATAAGGATCGGCTAACTCCGTGCC
AGCAGCCGCGGTAATACGGAGGATCCGAGCGTTATCCGGATTTATTGGGTTTAAAGGGTGCGTA
GGTGGCTTTTTAAGTCAGCGGTGAAAGTTTGTGGCTCAACCATAAAATTGCCGTTGAAACTGGA
GGGCTTGAGTATATTTGAGGTAGGCGGAATGCGTGGTGTAGCGGTGAAATGCATAGATATCACG
CAGAACTCCAATTGCGAAGGCAGCTTACTAAACTATAACTGACACTGAAGCACGAAAGCGTGGG
GATCAAACAGGATTAGATACCCTGGTAGTCCACGCAGTAAACGATGATTACTAGGAGTTTGCGA
TACACAGTAAGCTCTACAGCGAAAGCGTTAAGTAATCCACCTGGGGAGTACGCCGGCAACGGTG
AAACTCAAAGGAATTGACGGGGGCCCGCACAAGCGGAGGAACATGTGGTTTAATTCGATGATAC
GCGAGGAACCTTACCCGGGTTTGAACGTAAGTTGACCGGAGTGGAAACACTCTTTCTAGCAATA
GCAATTTACGAGGTGCTGCATGGTTGTCGTCAGCTCGTGCCGTGAGGTGTCGGCTTAAGTGCCA
TAACGAGCGCAACCCTTATCTTTAGTTACTAACAGGTCGAGCTGAGGACTCTAAAGAGACTGCC
AGCGTAAGCTGTGAGGAAGGTGGGGATGACGTCAAATCAGCACGGCCCTTACATCCGGGGCGAC
ACACGTGTTACAATGGTGGGGACAAAGGGCAGCTACCTGGCGACAGGATGCTAATCTCCAAACC
CCATCTCAGTTCGGATCGAAGTCTGCAACCCGACTTCGTGAAGCTGGATTCGCTAGTAATCGCG
CATCAGCCATGGCGCGGTGAATACGTTCCCGGGCCTTGTACACACCGCCCGTCAAGCCATGGGA
GTTGGGGGTACCTAAAGTCCGTAACCGCAAGGATCGGCCTAGGGTAAAACCGATGACTGGGGCT
AAGTCGTAACAAGGTAGCCGTACCGGAAGGTGCGGCTGGAACACCTCCTTT
Strain 20
Bacteroides uniformis 16S ribosomal RNA coding sequence (rDNA)
(SEQ ID NO: 20)
ATGAAGAGTTTGATCCTGGCTCAGGATGAACGCTAGCTACAGGCTTAACACATGCAAGTCGAGG
GGCAGCATGAACTTAGCTTGCTAAGTTTGATGGCGACCGGCGCACGGGTGAGTAACACGTATCC
AACCTGCCGATGACTCGGGGATAGCCTTTCGAAAGAAAGATTAATACCCGATGGCATAGTTCTT
CCGCATGGTGGAACTATTAAAGAATTTCGGTCATCGATGGGGATGCGTTCCATTAGGTTGTTGG
CGGGGTAACGGCCCACCAAGCCTTCGATGGATAGGGGTTCTGAGAGGAAGGTCCCCCACATTGG
AACTGAGACACGGTCCAAACTCCTACGGGAGGCAGCAGTGAGGAATATTGGTCAATGGACGAGA
GTCTGAACCAGCCAAGTAGCGTGAAGGATGACTGCCCTATGGGTTGTAAACTTCTTTTATACGG
GAATAAAGTGAGGCACGTGTGCCTTTTTGTATGTACCGTATGAATAAGGATCGGCTAACTCCGT
GCCAGCAGCCGCGGTAATACGGAGGATCCGAGCGTTATCCGGATTTATTGGGTTTAAAGGGAGC
GTAGGCGGACGCTTAAGTCAGTTGTGAAAGTTTGCGGCTCAACCGTAAAATTGCAGTTGATACT
GGGTGTCTTGAGTACAGTAGAGGCAGGCGGAATTCGTGGTGTAGCGGTGAAATGCTTAGATATC
ACGAAGAACTCCGATTGCGAAGGCAGCCTGCTGGACTGTAACTGACGCTGATGCTCGAAAGTGT
GGGTATCAAACAGGATTAGATACCCTGGTAGTCCACACAGTAAACGATGAATACTCGCTGTTTG
CGATATACAGTAAGCGGCCAAGCGAAAGCGTTAAGTATTCCACCTGGGGAGTACGCCGGCAACG
GTGAAACTCAAAGGAATTGACGGGGGCCCGCACAAGCGGAGGAACATGTGGTTTAATTCGATGA
TACGCGAGGAACCTTACCCGGGCTTGAATTGCAACTGAATGATGTGGAGACATGTCAGCCGCAA
GGCAGTTGTGAAGGTGCTGCATGGTTGTCGTCAGCTCGTGCCGTGAGGTGTCGGCTTAAGTGCC
ATAACGAGCGCAACCCTTATCGATAGTTACCATCAGGTGATGCTGGGGACTCTGTCGAGACTGC
CGTCGTAAGATGTGAGGAAGGTGGGGATGACGTCAAATCAGCACGGCCCTTACGTCCGGGGCTA
CACACGTGTTACAATGGGGGGTACAGAAGGCAGCTACACGGCGACGTGATGCTAATCCCGAAAG
CCTCTCTCAGTTCGGATTGGAGTCTGCAACCCGACTCCATGAAGCTGGATTCGCTAGTAATCGC
GCATCAGCCACGGCGCGGTGAATACGTTCCCGGGCCTTGTACACACCGCCCGTCAAGCCATGAA
AGCCGGGGGTACCTGAAGTGCGTAACCGCAAGGAGCGCCCTAGGGTAAAACTGGTGATTGGGGC
TAAGTCGTAACAAGGTAGCCGTACCGGAAGGTGCGGCTGGAACACCTCCTT

### EXAMPLES

### Example 1: Association of Bacterial Strains with Outcomes in Cancer

A live biotherapeutic product (LBP) consisting of 11 distinct nonpathogenic, nontoxigenic, commensal bacterial strains clonally derived from healthy donor stool was used. The LBP was formulated as an enteric capsule that contains approximately 400 mg of a mixture of 5×10⁷ CFU of lyophilized bacteria from each of 11 clonally-derived and distinct bacterial species for a total bacterial content of 5×10⁸ CFU per capsule, respectively.

CONSORTIUM-JO (NCT4208958) is an open-label, first-in-human study evaluating VE800 and nivolumab (PD-1 inhibitor) combination in patients with anti-PD-1/PD-L1 relapsed/refractory melanoma, anti-PD-1/PD-L1 naïve gastric/gastroesophageal junction (GEJ) adenocarcinoma, and anti-PD-1/PD-L1 naïve microsatellite-stable (MSS) colorectal cancer. In this study, the patients received oral (PO) vancomycin, 125 mg 4 times a day (QID) for 5 days as a pre-treatment. This pre-treatment was followed by a first cycle (4-week) consisting of the LBP (i.e., the enterically coated 11 strains capsule) orally (PO) of 5 capsules daily (QD) for the first 7 days only and then followed by 1 capsule daily (QD) continuously for the remaining three weeks. In other words, Day 1 is the day that the patients receive their first dose of the five capsules of LBP. In the next cycle of 4 weeks, the patients received one LBP capsule orally daily (QD) continuously for the four weeks. Nivolumab was administered intravenously (IV) at a dose of 480 mg as a 30-minute intravenous (IV) infusion on Day 1 of each 4-week treatment cycle. In other words, the patients received nivolumab on Day 1 and Day 29. Patients were dosed within a ±3-day window.

Human subjects with colorectal cancer, gastric cancer, or melanoma were administered a composition comprising 11 bacterial strains (“11-mix”), or a control composition that did not contain bacteria. The 11-mix contained bacterial strains Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis. Prior to administration, fecal samples from subjects were analyzed for the presence and abundance of each of the 11 bacterial strains. After administration, fecal samples were obtained from subjects and analyzed to measure the abundance of each of the 11 bacterial strains at days 1, 3, 7, 14, 21, 30, 58, and 86 post-administration of the LBP (i.e., the 11-mix).

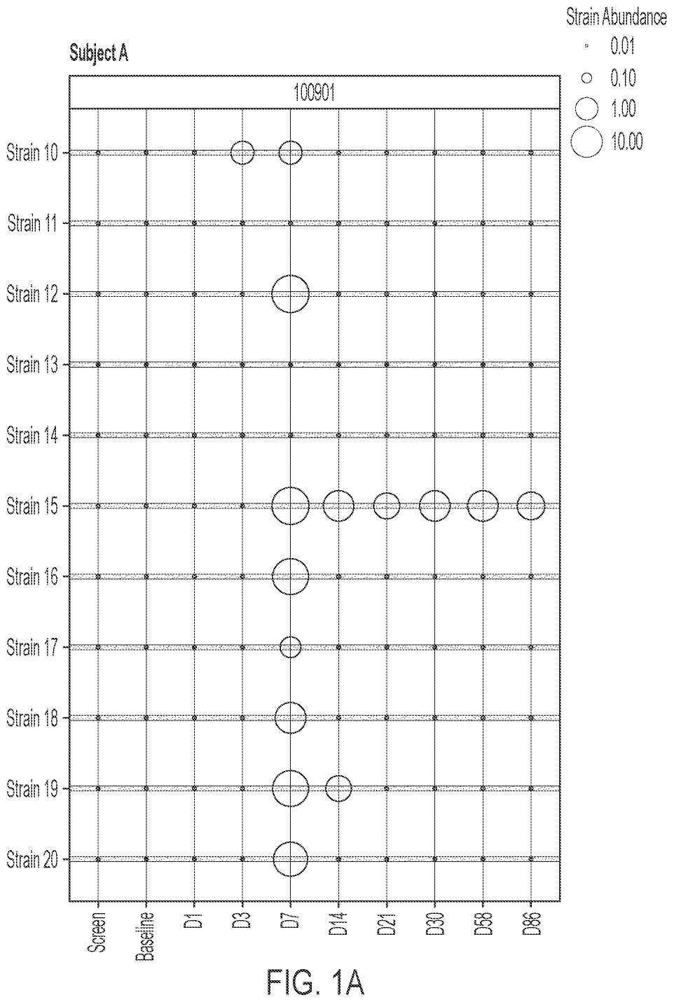

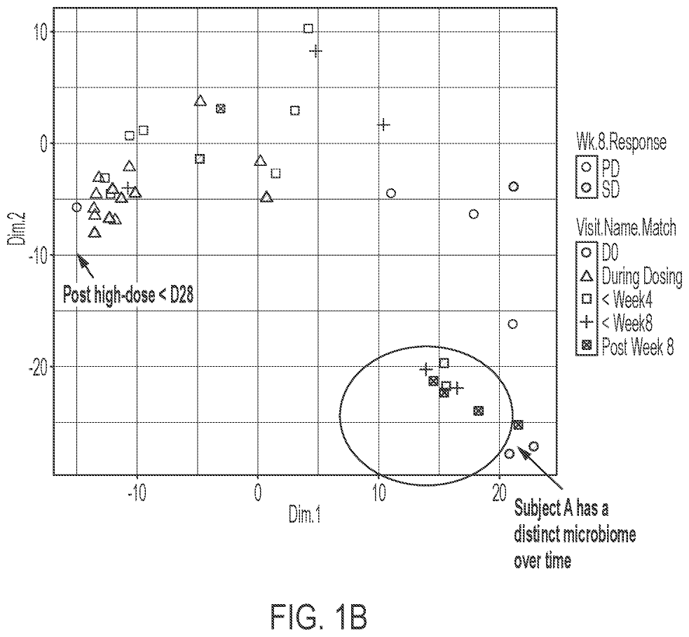

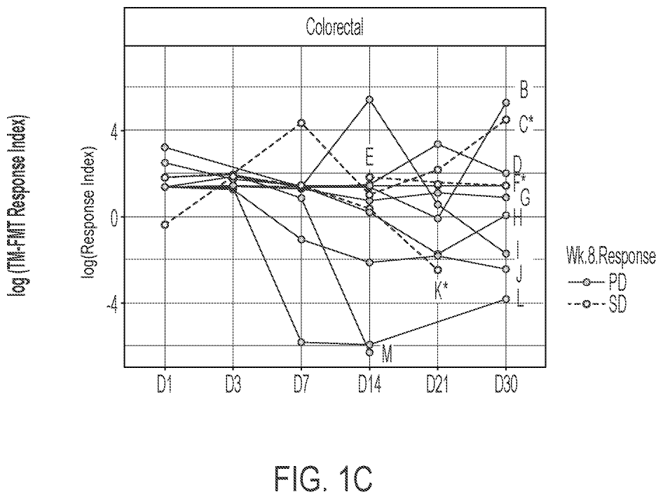

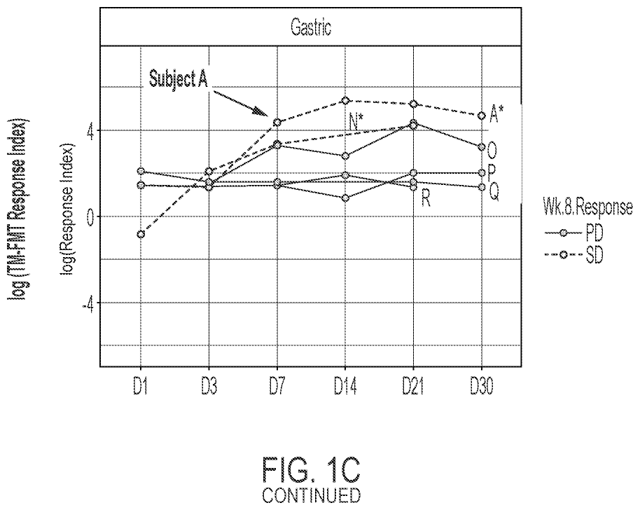

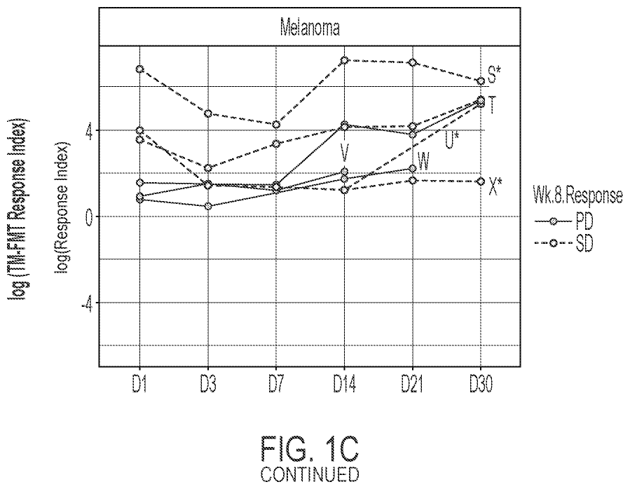

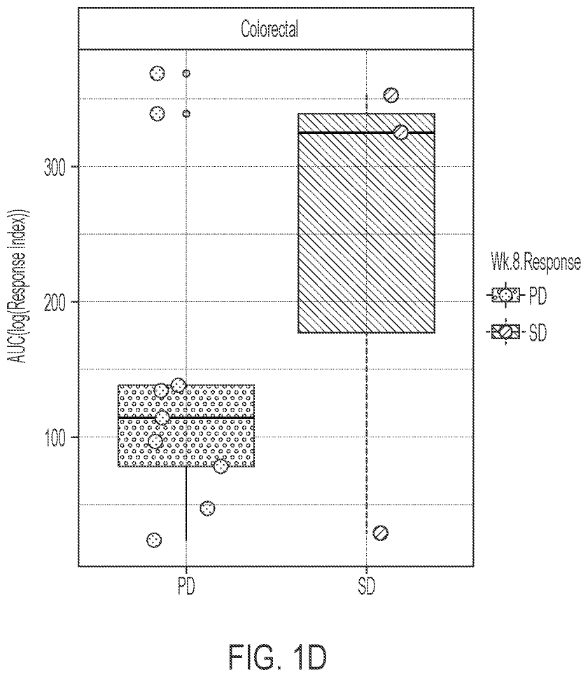

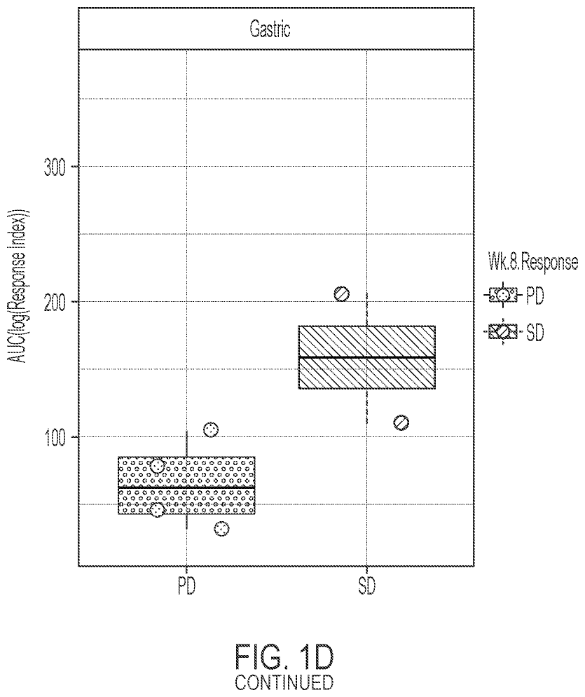

In one subject, referred to herein as Subject A, 8 of the 11 strains were detected at day 7 post-administration (i.e., 7 days after Day 1), but all but one of the strains of the 11-mix were lost by day 21 post-administration (FIG. 1A). Principal component analysis (PCA) was used to visualize relationships between the microbiomes of subjects over time and space. This analysis identified a distinct cluster of data points corresponding to the microbiome of Subject A at time points after day 28 post-administration (i.e., Day 28), indicating that Subject A had a distinct microbiome over time (FIG. 1B). Together, these results indicate that Subject A experienced a transient shift in the composition of their gut microbiome after administration of the 11-mix composition, but reverted to the baseline composition thereafter.

Subjects were also monitored over time for signs of cancer progression or recovery, as measured by a TM-FMT response index. Briefly, a response index is a diagnostic quantity computed from microbial taxa phenotypically associated with response to checkpoint therapy across a meta-analysis of existing microbiome-cancer translational medicine and FMT studies. It is defined as below:

$$( Bacteroides . thetaiotaomicron + Akkermansia muciniphila + Collinsella . aerofaciens + Bifido . longum + Bifido . adolescentis + Ruminococcus . gnavus + Faecalibacterium . prausnitzii ) \text{} / ( Anaerostipes . caccae + Bacteroides . fragilis )$$

The source data investigated to define the response index included the following:

Translational medicine studies: Patients underwent checkpoint therapy without an additional microbiome intervention. Meta-analysis of published and unpublished metagenomics datasets was carried out at Vedanta.

Unpublished datasets—2 translational medicine datasets (primarily melanoma, some lung cancer patients).

Published datasets: 1 translational medicine dataset in melanoma patients published in V. Gopalakrishnan et al., Science (2018) 359(6371): 97-103 (doi: 10.1126/science.aan4236 (2017)), incorporated herein by reference.

Through combined analysis of the above datasets, a pre-checkpoint baseline microbiome associated with response to checkpoint therapy was identified.

From this analysis, the following conclusions were made:

A high baseline abundance of in Akkermansia muciniphila and Bacteroides thetaiotaomicron is predictive of response to the LBP (i.e., the 11-mix).

A High baseline microbiome of Bacteroides fragilis is predictive of non-response.

Clinical studies: Published datasets of melanoma patients undergoing checkpoint therapy with fecal matter transfer (FMT).

Bifidobacterium longum, Collinsella aerofaciens, Faecalibacterium prausnitzii enriched in responders post-FMT (See, D. Davar, et al. Science (2021) 371.6529: 595-602)

Bifidobacterium adolescentis, Ruminococcus gnavus-enriched in responders post-FMT (See, E. N. Baruch et al., Science (2021) 371(6529): 602-609; (10.1126/science.abb5920 (2020))

Additional findings: Baseline microbiome of melanoma and colorectal cancer (CRC) patients with stable disease was found to be enriched in Akkermansia sp. and Bacteroides thetaiotaomicron. While baseline microbiome of non-responders was enriched in Bacteroides fragilis and Anaerostipes caccae.

From this, the following conclusion was made:

Dynamic enrichment of Bifidobacterium sp., Faecalibacterium sp., and Collinsella sp. post-dosing with FMT/LBP is predictive of response.

The response index was defined using cross-study results from the above and tested for ability to distinguish disease status in the clinical study described in this Example. While derived primarily from melanoma patients, the index distinguished progressive disease from stable disease in this clinical study with 80% accuracy across melanoma, gastric, and colorectal cancer (CRC) subjects (within first 4 weeks post start of the dosing of the LBP 11-mix and checkpoint therapy).

As shown in FIG. 1C, despite the reversion of their gut microbiome to its baseline state after transient colonization by strains of the 11-mix, Subject A exhibited the greatest improvement of subjects with gastric cancer (middle panel). FIG. 1D shows the response index from all three cohorts (colorectal cancer, gastric cancer, and melanoma) with progressive disease (pink, left bars) and stable disease (blue, right bars).

Based on the results above, the gut microbiome of Subject A was then investigated as a source of bacterial strains with potential activity against gastric cancer.

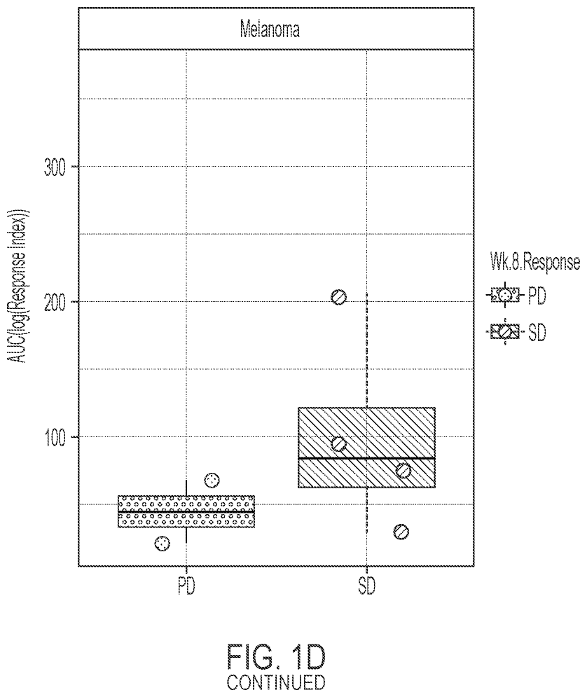

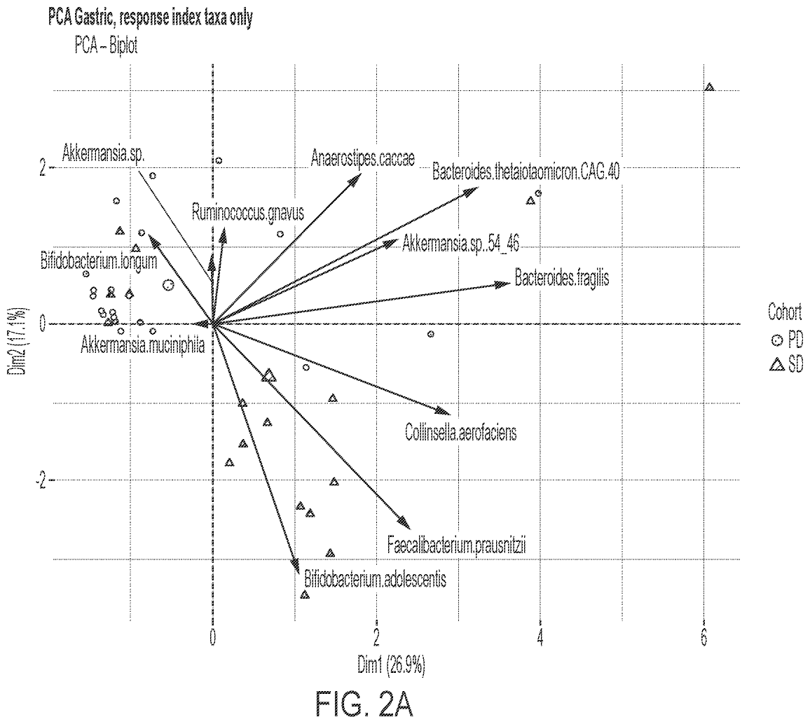

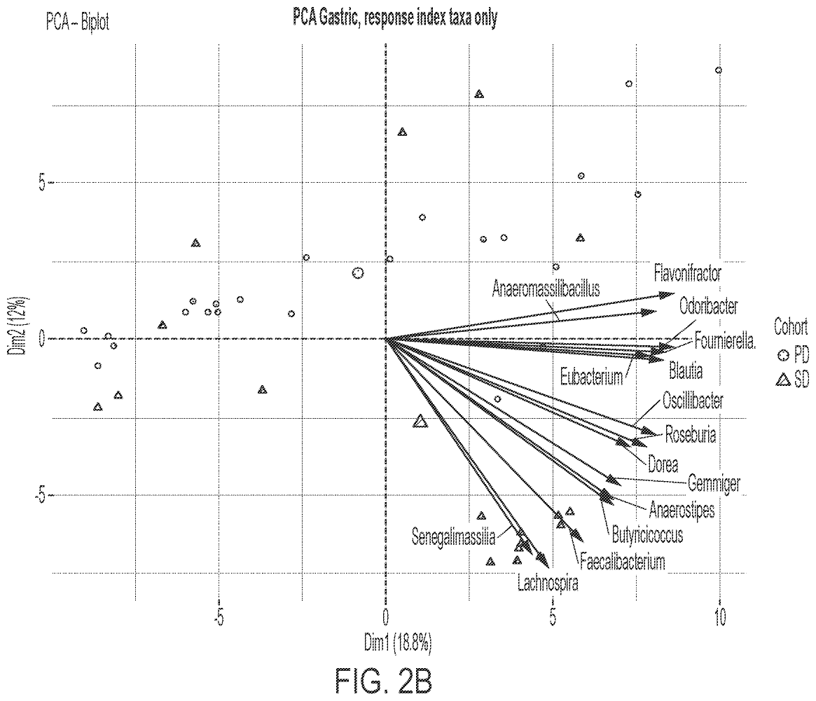

PCA was used to visualize the relationships of all bacterial taxa present in the microbiomes of subjects in the gastric cancer cohort. This analysis revealed that multiple genera of bacteria, including Faecalibacterium, Anaerostipes, Butyricicoccus, Dorea, Gemmiger, Lachnospira, Roseburia, and Oscillibacter, were positively correlated with the microbiome of Subject A, indicating that bacteria of these genera are associated with improvement in gastric cancer symptoms (FIG. 2B). PCA was also used to visualize relationships between the abundance of 10 bacterial species, which comprise a Response Index as an indicator of response to treatment, of the gut microbiomes of subjects in the gastric cancer cohort. Three species, Collinsella aerofaciens, Faecalibacterium prausnitzii, and Bifidobacterium adolescentis, were positively correlated with the microbiome of Subject A, indicating they are associated with improvement of gastric cancer symptoms (FIG. 2A). Three other species, Ruminococcus gnavus, Bacteroides fragilis, and Anaerostipes caccae, were negatively associated with the gut microbiome of Subject A.

### Example 2: Treatment of Cancer Using Purified Bacterial Strains

Subjects with cancer (e.g., gastric cancer) are treated by administration of a purified bacterial mixture comprising one or more bacterial strains, or a control composition containing no bacteria.

Group 1 (3-mix A): Collinsella aerofaciens, Faecalibacterium prausnitzii, and Bifidobacterium adolescentis;

Group 1A (3-mix A plus I): Collinsella aerofaciens, Faecalibacterium prausnitzii, and Bifidobacterium adolescentis plus one, two, three, or four additional strain(s) selected from Bifidobacterium longum, Ruminococcus gnavus, Akkermansia muciniphila, and Bacteroides thetaiotaomicron;

Group 1B (3-mix A plus II) Collinsella aerofaciens, Faecalibacterium prausnitzii, and Bifidobacterium adolescentis. plus one, two, three, four, five, six, seven, eight, or nine additional strain(s) selected from one of the following genera: Dorea, Lachnospira, Roseburia, Faecalibacterium, Blautia, Anaerostipes, Butyricicoccus, Gemmiger and Oscillibacter;

Group 2 (3-mix A+11-mix): Collinsella aerofaciens, Faecalibacterium prausnitzii, and Bifidobacterium adolescentis, as well as one, two, three, four, five, six, seven, eight, nine, ten, or eleven additional strain(s) selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis;

Group 2A (3-mix A plus I+11 mix): Collinsella aerofaciens, Faecalibacterium prausnitzii, and Bifidobacterium adolescentis plus one, two, three, or four additional strain(s) selected from Bifidobacterium longum, Ruminococcus gnavus, Akkermansia muciniphila, and Bacteroides thetaiotaomicron, as well as one, two, three, four, five, six, seven, eight, nine, ten, or eleven additional strain(s) selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis;

Group 2A (3-mix A plus II+11 mix): Collinsella aerofaciens, Faecalibacterium prausnitzii, and Bifidobacterium adolescentis. plus one, two, three, four, five, six, seven, eight, or nine additional strain(s) selected from one of the following genera: Dorea, Lachnospira, Roseburia, Faecalibacterium, Blautia, Anaerostipes, Butyricicoccus, Gemmiger and Oscillibacter, as well as one, two, three, four, five, six, seven, eight, nine, ten, or eleven additional strain(s) selected from Phascolarctobacterium faecium, Fusobacterium ulcerans, Bacteroides dorei, Bacteroides uniformis, Subdoligranulum sp., Paraprevotella xylaniphila, Parabacteroides johnsonii;

Group 3 (3-mix B): Ruminococcus gnavus, Bacteroides fragilis, and Anaerostipes caccae;

Group 4 (3-mix B+11-mix): Ruminococcus gnavus, Bacteroides fragilis, and Anaerostipes caccae, as well as one, two, three, four, five, six, seven, eight, nine, ten, or eleven additional strain(s) selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis;

Group 5 (11-mix only): Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis; and

Group 6 (negative control): No bacteria.

Subjects in each group are administered a composition comprising the specified bacterial strains. The subjects may be administered the composition orally, in a mixture of strains (e.g., in an enterically coated capsule) or each strain individually (e.g., each stain in an enterically coated capsule). At multiple time points after administration, stool samples are obtained from subjects, and the abundance of bacterial strains of the gut microbiome are quantified by DNA extraction and sequencing of 16S rDNA sequences present in stool.

Subjects are also monitored over time for cancer signs and symptoms, including tumor size and metastasis. Tumor size and/or volume is evaluated by imaging techniques such as CT scan or MRI. Metastasis is evaluated by similar imaging techniques to detect the presence of cancer cells, lesions, or tumors in other anatomical sites, as well as obtaining samples from anatomical sites via biopsy and detecting the presence of cancer cells by histology. The systemic induction of proliferation and/or accumulation of CD8⁺ T cells in subjects is evaluated by obtaining blood samples from subjects, isolating peripheral blood mononuclear cells (PBMCs) from blood, staining for multiple T cell markers including CD3 and CD8, and quantifying CD3⁺CD8⁺ T cells by flow cytometry. IFN-γ+ cells are identified by also permeabilizing cells and staining for the presence of IFN-γ in cells and detecting CD3⁺CD8⁺IFN-γ⁺ T cells. The accumulation of CD8⁺ T cells in the gut is evaluated by obtaining a sample of the gut of subjects via biopsy, preparing single-cell suspensions of the gut samples, and detecting CD3⁺CD8⁺ and/or CD3⁺CD8⁺IFN-γ⁺ T cells using similar staining and flow cytometry methods.

Any of the compositions recited above may be administered in conjunction with an immune checkpoint inhibitor, such as a PD-1 inhibitor, PD-L1 inhibitor, or CTLA-4 inhibitor, and the effects of the inhibitor(s) on gut microbiome composition, cancer progression, and CD8⁺ T cell induction may be evaluated as described above.

What is claimed is:

1. A composition comprising a purified bacterial mixture comprising one or more bacterial strains selected from the group consisting of Collinsella aerofaciens, Bifidobacterium adolescentis, and Faecalibacterium prausnitzii.

2. The composition of claim 1, wherein the purified bacterial mixture does not comprise Ruminococcus gnavus, Bacteroides fragilis, and Anaerostipes caccae.

3. The composition of claim 1 or 2, wherein the purified bacterial mixture comprises 2 or 3 of the bacterial strains.

4. The composition of any one of claims 1-3, wherein the purified bacterial mixture consists of Collinsella aerofaciens, Bifidobacterium adolescentis, and Faecalibacterium prausnitzii.

5. The composition of any one of claims 1-3, wherein the purified bacterial mixture further comprises one or more bacterial strains selected from the group consisting of Fusobacterium varium, Fusobacterium ulcerans; Fusobacterium sp., Eubacteium limosum, Geminger formicilis, Subdolinogranulum sp.; Ruminococcaceae bacterium; Ruthenibacterium lactatiformans, Phascolarctobacterium faecium, Phascolarctobacterium sp. CAG:207, Parabacteroides johnsonii, Paraprevotella xylaniphila, Parabacteroides distasonis, Alistipes senegalensis, Alistipes timonensis, Bacteroides dorei, Bacteroides fluxus, Phocaeicola dorei, Bacteroides_B dorei, Parabacteroides gordonii, Parabacteroides timonensis, and Bacteroides uniformis.

6. The composition of any one of claims 1, 2, or 5, wherein the purified bacterial mixture further comprises one or more bacterial strains selected from the group consisting of Bifidobacterium longum, Ruminococcus gnavus, Akkermansia municiniphila, Bacterioides thetaiotaomicron.

7. The composition of any one of claims 1, 2, 5, or 6, wherein the purified bacterial mixture further comprises one or more bacterial strains belonging to a bacterial genera selected from the group consisting of Dorea, Lachnospira, Roseburia, Faecalibacterium, Blautia, Anaerostipes, Butyricicoccus, Gemmiger and Oscillibacter.

8. A composition comprising a purified bacterial mixture comprising one or more bacterial strains selected from the group consisting of Collinsella aerofaciens, Bifidobacterium adolescentis, Faecalibacterium prausnitzii, Bifidobacterium longum, Ruminococcus gnavus, Akkermansia muciniphila, and Bacteroides thetaiotaomicron.

9. A composition comprising a purified bacterial mixture comprising one or more bacterial strains selected from the group consisting of Collinsella aerofaciens, Bifidobacterium adolescentis, Faecalibacteriium prausnitzii and bacterial species belonging to bacterial genera Dorea, Lachnospira, Roseburia, Faecalibacterium, Blautia, Anaerostipes, Butyricicoccus, Gemmiger, and Oscillibacter.

10. A composition comprising a purified bacterial mixture comprising Collinsella aerofaciens, Bifidobacterium adolescentis, Faecalibacteriium prausnitzii and one or more bacterial strains selected from Bifidobacterium longum, Ruminococcus gnavus, Akkermansia muciniphila, and Bacteroides thetaiotaomicron.

11. A composition comprising a purified bacterial mixture comprising Collinsella aerofaciens, Bifidobacterium adolescentis, Faecalibacteriium prausnitzii and one or more bacterial strains belonging to the bacterial genera selected from the group consisting of Dorea, Lachnospira, Roseburia, Faecalibacterium, Blautia, Anaerostipes, Butyricicoccus, Gemmiger, and Oscillibacter.

12. A composition comprising a purified bacterial mixture comprising Collinsella aerofaciens, Bifidobacterium adolescentis, Faecalibacteriium prausnitzii and one or more bacterial strains selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis.

13. A composition comprising a purified bacterial mixture comprising Collinsella aerofaciens, Bifidobacterium adolescentis, Faecalibacteriium prausnitzii;
one or more bacterial strains selected from Bifidobacterium longum, Ruminococcus gnavus, Akkermansia muciniphila, and Bacteroides thetaiotaomicron; and
one or more bacterial strains selected from Phascolarctobacterium faecium, Fusobacterium ulcerans, Bacteroides dorei, Bacteroides uniformis, Subdoligranulum sp., 5 Paraprevotella xylaniphila, Parabacteroides johnsonii, Alistipes sp., Parabacteroides gordonii, Eubacterum limosum, and Parabacteroides distasonis.

14. A composition comprising a purified bacterial mixture comprising Collinsella aerofaciens, Bifidobacterium adolescentis, Faecalibacteriium prausnitzii;
one or more bacterial strains belonging to the bacterial genera selected from the group consisting of Dorea, Lachnospira, Roseburia, Faecalibacterium, Blautia, Anaerostipes, Butyricicoccus, Gemmiger, and Oscillibacter; and
one or more bacterial strains selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis.

15. A composition comprising a purified bacterial mixture comprising Ruminococcus gnavus, Bacteroides fragilis, and Anaerostipes caccae.

16. A composition comprising a purified bacterial mixture comprising Ruminococcus gnavus, Bacteroides fragilis, and Anaerostipes caccae and one or more bacterial strains selected from Phascolarctobacterium faecium, Fusobacterium ulcerans, Bacteroides dorei, Bacteroides uniformis, Subdoligranulum sp., Paraprevotella xylaniphila, Parabacteroides johnsonii, Alistipes sp., Parabacteroides gordonii, Eubacterum limosum, and Parabacteroides distasonis.

17. A composition comprising a purified bacterial mixture comprising one or more bacterial strains comprising 16S rDNA sequences having at least 97% sequence identity to the nucleic acid sequences selected from the group consisting of SEQ ID NOs: 1-3.

18. The composition of claim 5, wherein the purified bacterial mixture does not comprise a bacterial strain comprising a 16S rDNA sequence having at least 97% sequence identity to the nucleic acid sequence selected from the group consisting of SEQ ID NO: 4-6.

19. The composition of claim 17 or 18, wherein the purified bacterial mixture comprises 2 or 3 of the bacterial strains.

20. The composition of any one of claims 17-19, wherein the purified bacterial mixture consists of bacterial strains comprising 16S rDNA sequences having at least 97% sequence identity to the nucleic acid sequences provided by SEQ ID NOs: 1-3.

21. The composition of any one of claims 17-19, wherein the purified bacterial mixture further comprises one or more bacterial strains comprising 16S rDNA sequences having at least 97% sequence identity to the nucleic acid sequences selected from the group consisting of SEQ ID NOs: 10-20.

22. The composition of any one of claims 17-19 or 21, wherein the purified bacterial mixture further comprises one or more bacterial strains comprising 16S rDNA sequences having at least 97% sequence identity to the nucleic acid sequences selected from the group consisting of SEQ ID NO: 4, 7, 8, and 9.

23. The composition of any one of claims 17-19, 21, or 22, wherein the purified bacterial mixture further comprises one or more bacterial strains belonging to a bacterial genus selected from the group consisting of Dorea, Lachnospira, Roseburia, Faecalibacterium, Blautia, Anaerostipes, Butyricicoccus, Gemmiger, and Oscillibacter.

24. A composition comprising a purified bacterial mixture comprising one or more bacterial strains comprising 16S rDNA sequences having at least 97% sequence identity to the nucleic acid sequences selected from the group consisting of SEQ ID NOs: 1-4 and 7-9.

25. A composition comprising a purified bacterial mixture comprising bacterial strains comprising 16S rDNA sequences having at least 97% sequence identity to the nucleic acid sequences of SEQ ID NOs: 1-3, and
one or more bacterial strains belonging to a bacterial genera selected from the group consisting of Dorea, Lachnospira, Roseburia, Faecalibacterium, Blautia, Anaerostipes, Butyricicoccus, Gemmiger, and Oscillibacter.

26. A composition comprising a purified bacterial mixture comprising bacterial strains comprising 16S rDNA sequences having at least 97% sequence identity to the nucleic acid sequences of SEQ ID NOs: 1-3;
one or more bacterial strains comprising 16S rDNA sequences having at least 97% sequence identity to the nucleic acid sequences of SEQ ID NOs: 4 and 7-9; and
one or more bacterial strains comprising 16S rDNA sequences having at least 97% sequence identity to the nucleic acid sequences of SEQ ID NOs: 10-20.

27. A composition comprising a purified bacterial mixture comprising bacterial strains comprising 16S rDNA sequences having at least 97% sequence identity to the nucleic acid sequences of SEQ ID NOs: 1-3;
one or more bacterial strains belonging to a bacterial genera selected from the group consisting of Dorea, Lachnospira, Roseburia, Faecalibacterium, Blautia, Anaerostipes, Butyricicoccus, Gemmiger, and Oscillibacter; and
one or more bacterial strains comprising 16S rDNA sequences having at least 97% sequence identity to the nucleic acid sequences of SEQ ID NOs: 10-20.

28. A composition comprising a purified bacterial mixture comprising bacterial strains comprising 16S rDNA sequences having at least 97% sequence identity to the nucleic acid sequences of SEQ ID NOs: 4-6.

29. A composition comprising a purified bacterial mixture comprising bacterial strains comprising 16S rDNA sequences having at least 97% sequence identity to the nucleic acid sequences of SEQ ID NOs: 4-6 and
one or more bacterial strains comprising 16S rDNA sequences having at least 97% sequence identity to the nucleic acid sequences of SEQ ID NOs: 10-20.

30. A composition comprising a purified bacterial mixture comprising bacterial strains comprising 16S rDNA sequences having at least 97% sequence identity to the nucleic acid sequences of SEQ ID NOs: 1-3, and
one or more bacterial strains comprising 16S rDNA sequences having at least 97% sequence identity to the nucleic acid sequences of SEQ ID NOs: 10-20.

31. A composition comprising a purified bacterial mixture comprising bacterial strains comprising 16S rDNA sequences having at least 97% sequence identity to the nucleic acid sequences of SEQ ID NOs: 1-3, and
one or more bacterial strains comprising 16S rDNA sequences having at least 97% sequence identity to the nucleic acid sequences selected from the group consisting of SEQ ID NOs: 4 and 7-9.

32. The composition of any one of claims 1-31, wherein one or more of the bacterial strains are not spore-formers.

33. The composition of any one of claims 1-32, wherein one or more of the bacterial strains are in spore form.

34. The composition of any one of claims 1-33, wherein one or more of the bacterial strains are in vegetative form.

35. The composition of any one of claims 1-32 or 34, wherein each of the bacterial strains is in vegetative form.

36. The composition of any one of claims 1-35, wherein the bacterial strains originate from one human donor.

37. The composition of any one of claims 1-35, wherein the bacterial strains originate from more than one human donor.

38. The composition of any one of claims 1-37, wherein the bacterial strains are lyophilized.

39. The composition of any one of claims 1-37, wherein the bacterial strains are spray-dried.

40. The composition of any one of claims 1-39, wherein the composition comprises between 1×10⁷ and 1×10¹⁰ colony forming units (CFUs) per bacterial strain.

41. The composition of any one of claims 1-40, wherein the composition further comprises one or more enteric polymers.

42. The composition of any one of claims 1-41, wherein the composition further comprises one or more anticancer agents.

43. The composition of claim 42, wherein the anticancer agent is a cancer immunotherapy agent.

44. The composition of claim 43, wherein the cancer immunotherapy agent is an immune checkpoint inhibitor.

45. The composition of claim 44, wherein the immune checkpoint inhibitor is a PD-1 inhibitor, a PD-L1 inhibitor, or a CTLA-4 inhibitor.

46. A pharmaceutical composition comprising the composition of any one of claims 1-45, and a pharmaceutically acceptable excipient.

47. The composition of any one of claims 1-46, wherein the composition is formulated in a capsule.

48. The composition of any one of claims 1-47, wherein the composition is formulated for oral administration.

49. The composition of any one of claims 1-47, wherein the composition is formulated for rectal administration.

50. The composition of any one of claims 1-49, wherein the pharmaceutical composition is formulated for delivery to the intestine.

51. The composition of any one of claims 1-50, wherein the composition is formulated for delivery to the colon.

52. A method of treating cancer, the method comprising administering to a subject in need thereof an effective amount of the composition of any one of claims 1-51.

53. The method of claim 52, wherein cancer is gastric cancer.

54. The method of claim 52 or 53, wherein the subject is a human subject.

55. The method of any one of claims 52-54, wherein the subject is administered one or more doses of an antibiotic prior to administration of the composition.

56. The method of any one of claims 52-55, wherein the composition is administered to the subject as one dose.

57. The method of any one of claims 52-56, wherein the composition is administered to the subject more than once.

58. The method of any one of claims 52-55 or 57, wherein the composition is administered to the subject as multiple doses.

59. The method of any one of claims 52-58, further comprising administering one or more anticancer agents.

60. The method of claim 59, wherein the anticancer agent is a chemotherapy agent.

61. The method of claim 59 or 60, wherein the anticancer agent is a cancer immunotherapy agent.

62. The method of claim 61, wherein the cancer immunotherapy agent is an immune checkpoint inhibitor.

63. The method of claim 62, wherein the immune checkpoint inhibitor is a PD-1 inhibitor, PD-L-1 inhibitor, or CTLA-4 inhibitor.

64. The method of any one of claims 52-63, further comprising administering to the subject a second composition comprising a purified bacterial mixture comprising one or more bacterial strains.

65. The method of claim 64, wherein the second compositions comprises one or more bacterial strains selected from the group consisting of Fusobacterium varium, Fusobacterium ulcerans; Fusobacterium sp., Eubacteium limosum, Geminger formicilis, Subdolinogranulum sp.; Ruminococcaceae bacterium; Ruthenibacterium lactatiformans, Phascolarctobacterium faecium, Phascolarctobacterium sp. CAG:207, Parabacteroides johnsonii, Paraprevotella xylaniphila, Parabacteroides distasonis, Alistipes senegalensis, Alistipes timonensis, Bacteroides dorei, Bacteroides fluxus, Phocaeicola dorei, Bacteroides_B dorei, Parabacteroides gordonii, Parabacteroides timonensis, and Bacteroides uniformis.


SEQUENCE LISTING (20 sequences)

SEQ ID NO: 1 - DNA, 831 bp, Collinsella aerofaciens
gattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctaggtgtggggggacgatccccccgtgccgcagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatatgggtgaagcgggggagaccccgtggccgagaggagcccatacaggtggtgcatggctgtcgtcagctcgtgtcgtgagatgttgggttaagtcccgcaacgagcgcaacccccgccgcgtgttgccatcgggtgatgccgggaacccacgcgggaccgccgccgtcaaggcggaggagggcggggacgacgtcaagtcatcatgccccttatgccctgggctgcacacgtgctacaatggccggtacagagggatgccaccccgcgagggggagcggatcccggaaagccggccccagttcggattgggggctgcaacccgcccccatgaagtcggagttgctagtaatcgcggatcagcatgccgcggtgaatgcgttcccgggccttgt

SEQ ID NO: 2 - DNA, 920 bp, Bifidobacterium adolescentis
attattgggcgtaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacactgtgaggtggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaacctttttggggggagccgtctaaggtgaga

SEQ ID NO: 3 - DNA, 935 bp, Faecalibacteriium prausnitzii
gcgcgctacacatgcaagtcgaacgagcgagagagagcttgctttctcgagcgagtggcgaacgggtgagtaacgcgtgaggaacctgcctcaaagagggggacaacagttggaaacgactgctaataccgcataagcccacgrcycggcatcgggtagagggaaaaggagcaatccgctttgagatggcctcgcgtccgattagctagttggtgaggtaayggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcctgc

SEQ ID NO: 4 - DNA, 787 bp, Ruminococcus gnavus
catgcaagtcgagcgaagcaccttgacggatttcttcggattgaagccttggtgactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgkcgggtggcaaagccattc

SEQ ID NO: 5 - DNA, 908 bp, Bacteroides fragilis
ggatttattgggtttaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaatcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggatcgtcctagggtaaaac

SEQ ID NO: 6 - DNA, 904 bp, Anaerostipes caccae
attactgggtgtaagggtgcgtaggtggcatggtaagtcagaagtgaaagcccggggcttaaccccgggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaacaggattagataccctggtagtccacgccgtaaacgatgaatactaggtgtcggggccgtagaggcttcggtgccgcagcaaacgcagtaagtattccacctggggagtacgttcgcaagaatgaaactcaaaggaattgacggggacccgcacaagcggtggagcatgtggtttaattcgaagcaacgcgaagaaccttacctggtcttgacatcccaatgaccgaaccttaacsggwtttttctttcgagacaktkgagacaggtggtgcatggttgtcgtcagctcgtgtcgtgagatgttgggttaagtcccgcaacgagcgcaacccctatctttagtagccagcatttaaggtgggcactctagagagactgccagggataacctggaggaaggtggggacgacgtcaaatcatcatgccccttatggccagggctacacacgtgctacaatggcgtaaacaaagggaagcgaagtcgtgaggcgaagcaaatcccagaaataacgtctcagttcggattgtagtctgcaactcgactacatgaagctggaatcgctagtaatcgtgaatcagaatgtcacggtgaatacgttcccgggtcttgtacacaccgcccgtcacaccatgggagtcagtaacgcccgaagtcagtgacccaaccgcatggaaggarctg

SEQ ID NO: 7 - DNA, 907 bp, Bacteroides thetaiotaomicron
ggatttattgggtttaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagccgtaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaact

SEQ ID NO: 8 - DNA, 931 bp, Bifidobacterium longum
cggattattgggcgtaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtg

SEQ ID NO: 9 - DNA, 889 bp, Akkermansia muciniphila
cgatgttcggatcactgggcgtaagcgtgcgtaggcggtttcgtaagtcgtgtgtgaaaggcgggggctcaacccccggactgcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtaaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgaagtat

SEQ ID NO: 10 - DNA, 1503 bp, Fusobacterium ulcerans
cgaagagtttgatcctggctcaggatgaacgctgacagaatgcttaacacatgcaagtctacttgatccttcgggtgaaggtggcggacgggtgagtaacgcgtaaagaacttgccttacagactgggacaacatttggaaacgaatgctaataccggatattatgattgggtcgcatgatctgattatgaaagctatatgcgctgtgagagagctttgcgtcccattagttagttggtgaggtaacggctcaccaagacgatgatgggtagccggcctgagagggtgaacggccacaaggggactgagacacggcccttactcctacgggaggcagcagtggggaatattggacaatggaccaaaagtctgatccagcaattctgtgtgcacgaagaagtttttcggaatgtaaagtgctttcagttgggaagaagtcagtgacggtaccaacagaagaagcgacggctaaatacgtgccagcagccgcggtaatacgtatgtcgcaagcgttatccggatttattgggcgtaaagcgcgtctaggcggcttagtaagtctgatgtgaaaatgcggggctcaaccccgtattgcgttggaaactgctaaactagagtactggagaggtaggcggaactacaagtgtagaggtgaaattcgtagatatttgtaggaatgccgatggggaagccagcctactggacagatactgacgctaaagcgcgaaagcgtgggtagcaaacaggattagataccctggtagtccacgccgtaaacgatgattactaggtgttgggggtcgaacctcagcgcccaagctaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcccaagaagttaacagagatgttttcgtgcctcttcggaggaacttggtgacaggtggtgcatggctgtcgtcagctcgtgtcgtgagatgttgggttaagtcccgcaacgagcgcaacccctttcgtatgttaccatcattaagttggggactcatgcgagactgcctgcgatgagcaggaggaaggtggggatgacgtcaagtcatcatgccccttatacgctgggctacacacgtgctacaatgggtagtacagagagctgcaaacctgcgagggtaagctaatctcataaaactattcttagttcggattgtactctgcaactcgagtacatgaagttggaatcgctagtaatcgcaaatcagctatgttgcggtgaatacgttctcgggtcttgtacacaccgcccgtcacaccacgagagttggttgcacctgaagtaacaggcctaaccgtaaggagggatgttccgagggtgtgattagcgattggggtgaagtcgtaacaaggtatccgtacgggaacgtgcggatggatcacctcctt

SEQ ID NO: 11 - DNA, 1521 bp, Eubacterium limosum
tattgagagtttgatcctggctcaggacgaacgctggcggtatgcttaacacatgcaagtcgaacgagaaggttttgatggatccttcgggtgatatcagaactggaaagtggcgaacgggtgagtaacgcgtgggtaacctgccctatggaaaggaatagcctcgggaaactgggagtaaagccttatattatggttttgtcgcatggcaagatcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaagaatgacggtacccaatgaggaagtcccggctaactacgtgccagcagccgcggtaatacgtaggggacaagcgttgtccggaatgactgggcgtaaagggcgcgtaggcggtctattaagtctgatgtgaaaggtaccggctcaaccggtgaagtgcattggaaactggtagacttgagtattggagaggcaagtggaattcctagtgtagcggtgaaatgcgtagatattaggaggaacaccagtggcgaaggcggcttgctggacaaatactgacgctgaggtgcgaaagcgtggggagcgaacaggattagataccctggtagtccacgccgtaaacgatgaatgctaggtgttggggaaactcagtgccgcagttaacacaataagcattccgcctggggagtacgaccgcaaggttgaaactcaaaggaattgacggggacccgcacaagcagcggagcatgtggtttaattcgaagcaacgcgaagaaccttaccaggtcttgacatcctctgacgagcctagagataggaagtttccttcgggaacagagagacaggtggtgcatggttgtcgtcagctcgtgtcgtgagatgttgggttaagtcccgcaacgagcgcaacccctgcctttagttgccagcattaagttgggcactctagagggactgccgtagacaatacggaggaaggtggggacgacgtcaaatcatcatgccccttatgacctgggctacacacgtgctacaatggtctgaacagagggccgcgaagccgcgaggtgaagcaaatcccttaaaacagatcccagttcggattgcaggctgcaactcgcctgcatgaagttggagttgctagtaatcgcggatcagaatgccgcggtgaatgcgttcccgggtcttgtacacaccgcccgtcacaccacgagagttggcaacacccgaagcctgtgagagaaccgtaaggactcagcagtcgaaggtggggctagtaattggggtgaagtcgtaacaaggtagccgtatcggaaggtgcggctggatcacctccttt

SEQ ID NO: 12 - DNA, 1526 bp, Subdoligranulum sp.
aatgaagagtttgatcctggctcaggacgaacgctggcggcgcgcctaacacatgcaagtcgaacggagctgttttctctgaagttttcggatggaagagagttcagcttagtggcgaacgggtgagtaacacgtgagcaacctgcctttcagtgggggacaacatttggaaacgaatgctaataccgcataagaccacagtgtcgcatggcacaggggtcaaaggatttatccgctgaaagatgggctcgcgtccgattagctagatggtgaggtaacggcccaccatggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgtcccaggggacgataatgacggtaccctgggaggaagcaccggctaactacgtgccagcagccgcggtaaaacgtagggtgcaagcgttgtccggaattactgggtgtaaagggagcgcaggcggattggcaagttgggagtgaaatctatgggctcaacccataaattgctttcaaaactgtcagtcttgagtggtgtagaggtaggcggaattcccggtgtagcggtggaatgcgtagatatcgggaggaacaccagtggcgaaggcggcctactgggcactaactgacgctgaggctcgaaagcatgggtagcaaacaggattagataccctggtagtccatgccgtaaacgatgattactaggtgtgggaggattgaccccttccgtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcggatgcatacctaagagattagggaagtccttcgggacatccagacaggtggtgcatggttgtcgtcagctcgtgtcgtgagatgttgggttaagtcccgcaacgagcgcaacccttatcgttagttactacgcaagaggactctagcgagactgccgttgacaaaacggaggaaggtggggatgacgtcaaatcatcatgccctttatgacctgggctacacacgtactacaatggctattaacagagagaagcgataccgcgaggtggagcaaacctcacaaaaatagtctcagttcggatcgcaggctgcaacccgcctgcgtgaagccggaattgctagtaatcgcggatcagcatgccgcggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtagccgtatcggaaggtgcggctggatcacctccttt

SEQ ID NO: 13 - DNA, 1557 bp, Phascolarctobacterium faecium
attggagagtttgatcctggctcaggacgaacgctggcggcgtgcctaacacatgcaagtcgaacggagaattttatttcggtagaattcttagtggcgaacgggtgagtaacgcgtaggcaacctaccctttagacggggacaacattccgaaaggagtgctaataccggatgtgatcatcttgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacggccgcaaggttgaaactcaaaggaattgacgggggcccgcacaagcggtggagtatgtggtttaattcgacgcaacgcgaagaaccttaccaaggcttgacattgattgaacgctctagagatagagatttcccttcggggacaagaaaacaggtggtgcatggctgtcgtcagctcgtgtcgtgagatgttgggttaagtcccgcaacgagcgcaacccctatcctatgttaccagcaagtaaagttggggactcatgggagactgccagggacaacctggaggaaggcggggatgacgtcaagtcatcatgccccttatgtcttgggctacacacgtactacaatggtcggaaacagagggaagcgaagccgcgaggcagagcaaaccccagaaacccgatctcagttcggatcgcaggctgcaacccgcctgcgtgaagtcggaatcgctagtaatcgcaggtcagcatactgcggtgaatacgttcccgggccttgtacacaccgcccgtcacaccacgaaagttggtaacacccgaagccggtgaggtaacctattaggagccagccgtctaaggtggggccgatgattggggtgaagtcgtaacaaggtagccgtatcggaaggtgcggctggatcacctccttt

SEQ ID NO: 14 - DNA, 1526 bp, Parabacteroides johnsonii
cgaagagtttgatcctggctcaggatgaacgctagcgacaggcttaacacatgcaagtcgaggggcatcatggtaagtagcaatacttattgatggcgaccggcgcacgggtgagtaacgcgtatgcaacttacctatcagagggggatagcccggcgaaagtcggattaatactccataaaacaggggttccgcatgggactatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggatgaaggatctatggtttgtaaacttcttttataggggaataaagtgtgggacgtgttccattttgtatgtaccctatgaataagcatcggctaactccgtgccagcagccgcggtaatacggaggatgcgagcgttatccggatttattgggtttaaagggtgcgtaggtggtaatttaagtcagcggtgaaagtttgtggctcaaccataaaattgccgttgaaactgggttacttgagtgtgtttgaggtaggcggaatgcgtggtgtagcggtgaaatgcatagatatcacgcagaactccaattgcgaaggcagcttactaaaccataactgacactgaagcacgaaagcgtgggtatcaaacaggattagataccctggtagtccacgcagtaaacgatgattactaggagtttgcgatacacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtcagaccgaccttgaaagaggttttctagcaatagctgattacgaggtgctgcatggttgtcgtcagctcgtgccgtgaggtgtcggcttaagtgccataacgagcgcaacccttatcactagttactaacaggttaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctctaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggtagccgtaccggaaggtgcggctggaacacctcctt

SEQ ID NO: 15 - DNA, 1319 bp, Paraprevotella xylaniphila
aataaagattaattggtaaaggatggggatgcgtcccattagcttgttggcggggtaacggcccaccaaggcgacgatgggtaggggttctgagaggaaggtcccccacattggaactgagacacggtccaaactcctacgggaggcagcagtgaggaatattggtcaatgggcgcgagcctgaaccagccaagtagcgtggaggacgacggccctacgggttgtaaactccttttataaggggataaagttggccatgtatggccatttgcaggtaccttatgaataagcatcggctaattccgtgccagcagccgcggtaatacggaagatgcgagcgttatccggatttattgggtttaaagggagcgtaggcgggcagtcaagtcagcggtcaaatggcgcggctcaaccgcgttccgccgttgaaactggcagccttgagtatgcacagggtacatggaattcgtggtgtagcggtgaaatgcttagatatcacgaggaactccgatcgcgcaggcattgtaccggggcattactgacgctgaggctcgaaggtgcgggtatcaaacaggattagataccctggtagtccgcacagtaaacgatgaatgcccgctgtcggcgacatagtgtcggcggccaagcgaaagcgttaagcattccacctggggagtacgccggcaacggtgaaactcaaaggaattgacgggggcccgcacaagcggaggaacatgtggtttaattcgatgatacgcgaggaaccttacccgggcttgaatcgcaggtgcatgggccggagacggccctttccttcgggactcctgcgaaggtgctgcatggttgtcgtcagctcgtgccgtgaggtgtcggcttaagtgccataacgagcgcaacccccctccccagttgccaccgggtaatgccgggcactttggggacactgccaccgcaaggtgcgaggaaggtggggatgacgtcaaatcagcacggcccttacgtccggggcgacacacgtgttacaatggggggtacagagggccgctgcccggtgacggttggccaatccctaaaacccctctcagttcggactggagtctgcaacccgactccacgaagctggattcgctagtaatcgcgcatcagccatggcgcggtgaatacgttcccgggccttgtacacaccgcccgtcaagccatgaaagccgggggtgcctgaagtccgtgaccgcgagggtcggcctagggtaaaaccggtgattggggctaagtcgtaacaaggtagccgtaccggaaggtgcggctggaacacctccttt

SEQ ID NO: 16 - DNA, 1521 bp, Parabacteroides distasonis
cgaagagtttgatcctggctcaggatgaacgctagcgacaggcttaacacatgcaagtcgaggggcagcacaggtagcaataccgggtggcgaccggcgcacgggtgagtaacgcgtatgcaacttgcctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtaaagctgaggactctggtgggactgccagcgtaagctgcgaggaaggcggggatgacgtcaaatcagcacggcccttacatccggggcgacacacgtgttacaatggcgtggacaaagggaagccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgcgaggatcggcctagggtaaaactggtgactggggctaagtcgtaacaaggtagccgtaccggaaggtgcggctggaacacctccttt

SEQ ID NO: 17 - DNA, 1525 bp, Alistipes sp.
atggagagtttgatcctggctcaggatgaacgctagcggcaggcctaacacatgcaagtcgaggggcagcgggattgaagcttgcttcagttgccggcgaccggcgcacgggtgcgtaacgcgtatgcaacctacccataacagggggataacactgagaaatcggtactaatatcccataacatcaagaggggcatcccttttggttgaaaactccggtggttatggatgggcatgcgttgtattagctagttggtgaggtaacggctcaccaaggcgacgatacatagggggactgagaggttaaccccccacattggtactgagacacggaccaaactcctacgggaggcagcagtgaggaatattggtcaatggacgcaagtctgaaccagccatgccgcgtgcaggatgacggctctatgagttgtaaactgcttttgtacgagggtaaacccggatacgtgtatccggctgaaagtatcgtacgaataaggatcggctaactccgtgccagcagccgcggtaatacggaggattcaagcgttatccggatttattgggtttaaagggtgcgtaggcggtttgataagttagaggtgaaataccggtgcttaacaccggaactgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggtggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctacccagtgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggctaagtcgtaacaaggtagccgtaccggaaggtgcggctggaacacctccttt

SEQ ID NO: 18 - DNA, 1528 bp, Bacteroides dorei
atgaagagtttgatcctggctcaggatgaacgctagctacaggcttaacacatgcaagtcgaggggcagcatggtcttagcttgctaaggctgatggcgaccggcgcacgggtgagtaacacgtatccaacctgccgtctactcttggccagccttctgaaaggaagattaatccaggatgggatcatgagttcacatgtccgcatgattaaaggtattttccggtagacgatggggatgcgttccattagatagtaggcggggtaacggcccacctagtcaacgatggataggggttctgagaggaaggtcccccacattggaactgagacacggtccaaactcctacgggaggcagcagtgaggaatattggtcaatgggcgatggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcactcgaatgatccggaaacggttcagctagcaatagcgagtgtgaaggtgctgcatggttgtcgtcagctcgtgccgtgaggtgtcggcttaagtgccataacgagcgcaacccttgttgtcagttactaacaggtgatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccctaaaacccctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggatcgccctagggtaaaactggtgactggggctaagtcgtaacaaggtagccgtaccggaaggtgcggctggaacacctcctt

SEQ ID NO: 19 - DNA, 1523 bp, Parabacteroides gordonii
cgaagagtttgatcctggctcaggatgaacgctagcgacaggcttaacacatgcaagtcgaggggcagcaggaagtagcaatactttgctggcgaccggcgcacgggtgagtaacgcgtatgcaacctacctatcagagggggataacccggcgaaagtcggactaataccgcataaaacaggggtcccgcatgggaatatttgttaaagatttattgctgatagatgggcatgcgttccattagatagttggtgaggtaacggctcaccaagtcttcgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggttcgtaaacttcttttataggggaataaagtgcaggacgtgtcctgttttgtatgtaccctatgaataaggatcggctaactccgtgccagcagccgcggtaatacggaggatccgagcgttatccggatttattgggtttaaagggtgcgtaggtggctttttaagtcagcggtgaaagtttgtggctcaaccataaaattgccgttgaaactggagggcttgagtatatttgaggtaggcggaatgcgtggtgtagcggtgaaatgcatagatatcacgcagaactccaattgcgaaggcagcttactaaactataactgacactgaagcacgaaagcgtggggatcaaacaggattagataccctggtagtccacgcagtaaacgatgattactaggagtttgcgatacacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtaagttgaccggagtggaaacactctttctagcaatagcaatttacgaggtgctgcatggttgtcgtcagctcgtgccgtgaggtgtcggcttaagtgccataacgagcgcaacccttatctttagttactaacaggtcgagctgaggactctaaagagactgccagcgtaagctgtgaggaaggtggggatgacgtcaaatcagcacggcccttacatccggggcgacacacgtgttacaatggtggggacaaagggcagctacctggcgacaggatgctaatctccaaaccccatctcagttcggatcgaagtctgcaacccgacttcgtgaagctggattcgctagtaatcgcgcatcagccatggcgcggtgaatacgttcccgggccttgtacacaccgcccgtcaagccatgggagttgggggtacctaaagtccgtaaccgcaaggatcggcctagggtaaaaccgatgactggggctaagtcgtaacaaggtagccgtaccggaaggtgcggctggaacacctccttt

SEQ ID NO: 20 - DNA, 1523 bp, Bacteroides uniformis
atgaagagtttgatcctggctcaggatgaacgctagctacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtggaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcctgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggtgatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaactggtgattggggctaagtcgtaacaaggtagccgtaccggaaggtgcggctggaacacctcctt